(19) United States
(12) Patent Application Publication
(10) Pub. No.: US 2026/0250572 A1
(43) Pub. Date: Aug. 27, 2026

(54) COMPOUNDS

(71) Applicant: Shoei Chemical Inc., Tokyo (JP)

(72) Inventors: IGOR COROPCEANU, Milpitas, CA (US); CHRISTIAN JUSTUS IPPEN, Cupertino, CA (US); ALAIN CHRISTOPHER BARRON, Redwood City, CA (US)

(21) Appl. No.: 19/159,220

(22) PCT Filed: Feb. 23, 2024
(86) PCT No.: PCT/US2024/017077

§ 371 Date: Aug. 22, 2025

Related U.S. Application Data
(60) Provisional application No. 63/448,207, filed on Feb. 24, 2023.

(51) Int. Cl. C09K 11/62; C07C 229/16

(52) U.S. Cl. CPC C09K 11/621; C07C 229/16

(57) ABSTRACT

Disclosed are examples of chemical compounds useful as ligands for photoluminescent particles, such as nanoparticles, populations of the particles comprising such ligands, films and other compositions comprising the ligated particles, and methods of synthesis.

### BACKGROUND

Nanostructures, e.g., quantum dots (QDs), which form colloids in common solvents, find application in device fabrication via solution processes, e.g., inkjet printing.

In order to disperse QDs in ultra-violet- (UV-) curable ink formulations, the native non-polar ligands may need to be replaced with more polar species. While commercially available chemicals such as Jeffamine® (polyetheramines based upon a predominantly polyethylene glycol (PEG) backbone, collectively “Jeffamine”, available from Huntsman Corporation of Woodlands, Texas) can assist dispersion QDs in commonly used monomer systems, such ligands may not be best suited to optimize the thermal and air stability of QDs. One possible cause of concern in the case of Jeffamine is the presence of ether linkages close to the QD surface. Such groups have an abstractable hydrogen which makes them prone to radical formation, which in turn can result in radical mediated damage to the QD surface. Indeed, in the case of QDs functionalized with Jeffamine, the quantum yield of QDs can rapidly degrade even in yellow light when exposed to air. Other candidate ligand systems, e.g., PEG-280 (PEG with an average molecular weight of 280 atomic mass units), also frequently possess such ether groups close to the amine anchoring group, causing the same issue.

There remains a need for ligands which can assist dispersion of QDs in polar solutions, e.g., polar UV-curable monomers, and/or which at least partly reduce degradation of QDs by thermal and/or oxidative processes.

### SUMMARY

One aspect of this disclosure relates to a class of chemical compounds useful as ligands for photoluminescent particles, such as nanoparticles. Other aspects relate to populations of the particles comprising such ligands, to films and other compositions comprising the ligated particles, and to methods of synthesis.

This Summary is provided in order to introduce in simplified form a selection of concepts that are further described in the Detailed Description. This Summary is not intended to identify key features or essential features of the claimed subject matter, nor is it intended to be used to limit the scope of the claimed subject matter. The claimed subject matter is not limited to implementations that solve any disadvantages noted in any part of this disclosure.

### BRIEF DESCRIPTION OF THE DRAWINGS

FIG. 1 is a line graph showing the Fourier transform infrared (FT-IR) spectrum of 4-hydroxybutylacrylate (HBA) alone and mixed with a 1:1 molar ratio of (1,3-bis(aminomethyl)cyclohexane) (CHBMA) after 1 minute and 5 minutes of mixing.

FIG. 2 is a line graph showing the proton nuclear magnetic resonance spectrum (¹H NMR) of the adduct formed between HBA and CHBMA (Compound 15), with an inset showing the disappearance of the resonances associated with the double bond of HBA.

FIG. 3 is a line graph showing the FT-IR spectrum of AgInGaS/GaS quantum dots with a combination of Compounds 15 and 22 as ligands.

FIG. 4 is a ¹H NMR spectrum of AgInGaS/GaS quantum dots with a combination of Compounds 15 and 22 as ligands.

FIG. 5 is an example box plot showing the photoconversion efficiency (PCE) of nanostructure films with and without hydroxyl functional groups present in the ligands of the nanostructures.

FIG. 6 is a line graph showing the proton nuclear magnetic resonance spectrum (¹H NMR) of the adduct formed between CHBMA and AHPMA (Compound 113).

FIG. 7 is a line graph showing the proton nuclear magnetic resonance spectrum (¹H NMR) of AIGS QDs with Compound 113 as a ligand.

FIG. 8 shows FTIR spectra of AHPMA, a full ink liquid printable composition including acylate monomers and AIGS QDs with a CAH ligand, and the final film produced by curing the film.

FIG. 9 shows schematically a side cross-section of an example light source.

FIGS. 10A and 10B show aspects of example display devices.

FIG. 11 shows aspects of an example computer system.

### DETAILED DESCRIPTION

Compounds according to examples herein can ligate to luminescent nanostructures. In examples these ligands assist dispersion of such nanostructures in polar solutions, e.g., polar UV-curable monomers. The compound structure may also or alternatively at least partly reduce degradation of the nanostructures by thermal and/or oxidative processes by spacing reactive sites in the ligand from the nanostructure surface. Some examples herein may include compounds which are capable of hydrogen bonding with other ligands on the nanostructure surface which may allow the ligands to further reduce degradation of the nanostructures by thermal and/or oxidative processes.

Further, compounds according to examples herein are obtainable by Michael addition reactions. Pairs of reagents comprising of a Michael-donor compound and a Michael-acceptor compound can be selected to generate a compound or compounds which, on ligation, provide a nanostructure with, e.g., desired polarity and resistance to degradative processes.

Some examples herein relate to a ligand which forms cross-links with a monomer of a carrier on polymerization of the monomer—e.g., by UV or heat. This is thought to lock the ligand and nanostructure in place. This may reduce aggregation of the nanostructures, increasing quantum yield of a device in use. This cross-linking may also reduce exposure of the nanostructure to thermal and/or oxidative degradation.

Unless defined otherwise, all technical and scientific terms used herein have the same meaning as commonly understood by one of ordinary skill in the art in the relevant field.

As used in this specification and the appended claims, the singular forms “a,” “an,” and “the” include plural referents unless the context clearly dictates otherwise. Thus, for example, reference to “a nanostructure” may include a plurality of such nanostructures, and the like.

The term “about” as used herein indicates the value of a given quantity varies by ±10% of the value. For example, “about 100 nm” encompasses a range of sizes from 90 nm to 110 nm, inclusive.

The term nanostructure as used herein refers for example to a structure having at least one region or characteristic dimension with a dimension of less than about 500 nm. In some examples the nanostructure has a dimension of less than about 200 nm, less than about 100 nm, less than about 50 nm, less than about 20 nm, or less than about 10 nm. Typically, the region or characteristic dimension will be along the smallest axis of the structure. Examples of such nanostructures include nanowires, nanorods, nanotubes, branched nanostructures, nanodots, quantum dots (QDs), nanoparticles, and the like. In some examples, each of the three orthogonal dimensions of the nanostructure has a dimension of less than about 500 nm, less than about 200 nm, less than about 100 nm, less than about 50 nm, less than about 20 nm, of less than about 10 nm.

The term “quantum dot” or “QD” as used herein refers for example to nanostructures that arc substantially monocrystalline (e.g., comprising a single crystal). For example, a QD may have a core-shell structure; the core is substantially monocrystalline and may have one or more shells thereon. A QD, e.g., has at least one region or characteristic dimension with a dimension of less than about 500 nm, and down to the order of less than about 1 nm. In some examples, the QDs have a maximum dimension of between about 2 nm and about 30 nm. Quantum dots described herein can be considered fluorescent semiconductor structures each with a semiconductor crystallite with a diameter less than or equal to twice the Bohr radius of an exciton inducible in the semiconductor crystallite. Such a radius results in quantum confinement of the exciton when induced in the semiconductor crystallite. The Bohr radius depends on the elemental composition of the semiconductor crystallite. For example, the Bohr radius of cadmium selenide (CdSe) is 5.4 nanometres, so a quasi-spherical CdSe semiconductor crystallite is a quantum dot if its radius is less than 5.4 nanometres. Further examples include: zinc selenide telluride (ZnSeTe) crystallites with a diameter between 4 and 5 nanometres, which are blue-light emitting quantum dots; indium phosphide (InP) crystallites with a diameter between 2 and 2.5 nanometres, which are green-light emitting quantum dots; and InP crystallites with a diameter between 2.8 and 3.5 nanometres, which are red-light emitting quantum dots. The quantum confinement of excitons results in fluorescence. Quantum confinement may be inducible in three dimensions, two dimensions (a quantum wire), or one dimension (a quantum well). Other morphologies of the semiconductor crystallite are envisaged such as cuboids or tetrahedrons. The quantum dots may comprise at least one of the following alloys: a III-V semiconductor, a II-VI semiconductor, zinc telluride selenide (ZnTeSe), zinc telluride (ZnTe), zinc selenide (ZnSe), zinc sulfide (ZnS), indium phosphide (InP), indium gallium phosphide (InGaP), indium arsenide (InAs), indium zinc phosphide (InZnP), indium arsenide (InAs), indium arsenide phosphide (InAsP), indium gallium arsenide phosphide (InGaAsP), silver indium gallium sulphide (AgInGaS or AIGS), copper indium sulfide (CuInS or CIS), copper indium gallium selenide (CuInGaSe or CIGS), cadmium selenide (CdSe), cadmium sulfide (CdS), cadmium telluride (CdTe), cadmium selenide telluride (CdSeTe), cadmium zinc selenide (CdZnSe), molybdenum sulphide (MoS), or an alloy thereof. The ratio(s) between the elements of alloys is not indicated, and various ratios are envisaged within as the skilled person will appreciate. The quantum dots may each comprise a core-shell structure with at least one shell on a core, the diameter of the core corresponding with, or less than, 2× the Bohr radius. The shell, for example, is a metal sulfide and/or a metal oxide. Example core-shell structures may be formed from CdSe(core)/CdS/ZnS, InP(core)/ZnSe/ZnS or AgInGaS(core)/GaS. The quantum dots may be functionalised with at least one ligand. A shell and/or ligand functionalisation may enhance the properties of the quantum dots such as the quantum yield, thermal stability and/or photo-stability. The quantum dots may be encapsulated to, for example, reduce the toxicity of the quantum dots. Many encapsulants are envisaged, such as a silane or a metal oxide, as the skilled person will appreciate.

The optical properties of quantum dots can be influenced by their particle size, chemical composition, and/or surface composition, and can be determined by suitable optical testing available in the art. The ability to tailor the nanocrystal size, e.g., in the range between about 1 nm and about 15 nm, enables photoemission coverage in the entire optical spectrum to offer great versatility in color rendering.

A “ligand” is in examples a compound or molecule capable of interacting with one a surface of a nanostructure, e.g., through covalent, ionic, van der Waals, dative bonding or coordination or other molecular interactions with the surface of the nanostructure. In some examples, the ligand may interact by covalent, ionic, or dative bonding.

A “ligand corona” is for example a plurality of ligands ligated to a luminescent nanostructure, such that the nanostructure is substantially surrounded by ligands, e.g., 50% or more of the nanostructure is surrounded by ligands.

“Quantum yield” is for example the ratio of the rate of photon emission to the rate of photon absorption for a photoluminescent specimen.

A “luminescence-wavelength peak” is for example the wavelength of a luminescence maximum in a luminescence spectrum. The luminescence maximum may be a local maximum and/or a global maximum.

The term “Michael-donor” as used herein refers to a nucleophile which can undergo a Michael-addition reaction with a Michael-acceptor. Suitable compounds include those with thiol or amino functional groups.

The term “Michael-acceptor” as used herein refers to a compound with an unsaturated electrophilic group (alkene). Typically this may be an α,β-unsaturated carbonyl.

The terms “Michael reaction”, “Michael addition”, “Michael-addition reaction” and the like refer to a reaction between a Michael-donor and a Michael-acceptor to produce a Michael adduct by creating a donor-carbon bond at the acceptor's β-carbon. The term includes aza-Michael reactions (nitrogen nucleophile), oxa-Michael reactions (oxygen nucleophile) and thia-Michael reactions (sulphur nucleophile).

The term “halo” or “halogen” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as herein used by itself or as part of another group refers to —OH.

The term “phenoxy” as used herein by itself or as part of another group refers to an oxygen radical that is connected to a phenyl group, i.e., a C₆ aryl. The term phenoxy includes phenoxy groups that are substituted at positions on the phenyl ring with one, two, or three groups selected from C₁-C₆ alkyl, C₁-C₆ alkoxy, hydroxy, halo, nitro, cyano, and amino. In some examples, the phenoxy has the following structure:

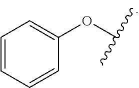 [SMILES: *Oc1ccccc1]

The term “amino” as used by itself or as part of another group refers to a radical of the formula —NR^{a}R^{b}, wherein R^{a} and R^{b} are independently hydrogen or alkyl. In one example, the amino is —NH₂. In another example, the amino is an “alkylamino,” i.e., an amino group wherein R^{a} is C₁₋₆ alkyl and R^{b} is hydrogen. In one example, R^{a} is C₁-C₄ alkyl. Non-limiting example alkylamino groups include —N(H)CH₃ and —N(H)CH₂CH₃. In another example, the amino is a “dialkylamino,” i.e., an amino group wherein R^{a} and R^{b} are each independently C₁₋₆ alkyl. In one example, R^{a} and R^{b} are each independently C₁-C₄ alkyl. Non-limiting Example dialkylamino groups include —N(CH₃)₂ and —N(CH₃)CH₂CH(CH₃)₂.

The term “alkyl” as used herein by itself or as part of another group refers to a straight- or branched-chain aliphatic hydrocarbon monovalent radical containing one to twelve carbon atoms, i.e., a C₁-C₁₂ alkyl, or the number of carbon atoms designated, e.g., C₁-C₃ alkyl such as methyl, ethyl, propyl, or isopropyl; a C₁-C₄ alkyl such as methyl, ethyl, propyl, isopropyl, n-butyl, isobutyl, sec-butyl, or t-butyl; and so on. In one example the alkyl is a straight-chain alkyl. In another example, the alkyl is a branched-chain alkyl. In one example, the alkyl is a C₁-C₈ alkyl. In another example, the alkyl is a C₁-C₆ alkyl. In another example, the alkyl is a C₁-C₄ alkyl. In another example, the alkyl is a C₁-C₃ alkyl. Non-limiting Example C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl. The term alkyl also includes alkyl groups that are substituted with one, two, or three groups selected from C₁-C₆ alkyl, C₁-C₆ alkoxy, hydroxy, halo, nitro, cyano, and amino.

The term “alkylene” refers to a divalent radical corresponding to a monovalent alkyl group as defined above. In some particular examples, the alkylene corresponds to one of the list of alkyl groups provided above.

The term “cycloalkyl” as used herein by itself or as part of another group refers to saturated and partially unsaturated, e.g., containing one or two double bonds, monocyclic, bicyclic, or tricyclic aliphatic hydrocarbon monovalent radical containing three to twelve carbon atoms, i.e., a C₃-C₁₂ cycloalkyl, or the number of carbons designated, e.g., a C₃-C₆ cycloalkyl such a cyclopropyl, cyclobutyl, cyclopentyl, and cyclohexyl. In one example, the cycloalkyl is bicyclic, i.e., it has two rings. In another example, the cycloalkyl is monocyclic, i.e., it has one ring. In another example, the cycloalkyl is a C₃-C₈ cycloalkyl. In another example, the cycloalkyl is a C₃-C₆ cycloalkyl. In another example, the cycloalkyl is a C₅ cycloalkyl, i.e., cyclopentyl. In another example, the cycloalkyl is a C₆ cycloalkyl, i.e., cyclohexyl. Non-limiting Example C₃-C₁₂ cycloalkyl groups include cyclopropyl, cyclobutyl, cyclopentyl, cyclohexyl, cycloheptyl, cyclooctyl, norbornyl, decalin, adamantyl, and cyclohexenyl. The term cycloalkyl also includes cycloalkyl groups that are substituted with one, two, or three groups selected from C₁-C₆ alkyl, C₁-C₆ alkoxy, hydroxy, halo, nitro, cyano, and amino. In one example, the cycloalkyl is bicyclo[2.2.1]heptanyl and is substituted with three methyl groups, e.g., to form 1,7,7-trimethylbicyclo[2.2.1]heptanyl. In another example, the cycloalkyl is bicyclo[2.2.1]heptanyl and is substituted with two aminomethyl groups, e.g., to form 2,3-(bisaminomethyl)bicyclo[2.2.1]heptanyl.

The term “cycloalkylene” refers to a divalent radical corresponding to a monovalent cycloalkyl group as defined above. In some particular examples, the cycloalkylene corresponds to one of the list of cycloalkyl groups provided above.

The term “heterocyclyl” as used herein by itself or as part of another group refers to saturated and partially unsaturated, e.g., containing one or two double bonds, monocyclic, bicyclic, or tricyclic monovalent radical groups containing three to eighteen ring members, i.e., a 3- to 18-membered heterocyclyl, comprising one, two, three, or four heteroatoms. Each heteroatom is independently oxygen, sulfur, or nitrogen. Each sulfur atom may be independently oxidized to give a sulfoxide, i.e., S(═O), or sulfone, i.e., S(═O)₂. The term heterocyclyl includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one. The term heterocyclyl also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one. In some examples, heterocyclyl is a 6-membered ring comprising two nitrogen atoms. The term heterocyclyl also includes heterocyclyl groups that are substituted with one, two, or three groups selected from C₁-C₆ alkyl, C₁-C₆ alkoxy, hydroxy, halo, nitro, cyano, and amino.

The term “heterocyclylene” refers to a divalent radical corresponding to a monovalent heterocyclyl group as defined above. In some particular examples, the heterocyclylene corresponds to one of the list of heterocyclyl groups provided above.

The term “aryl” as used herein by itself or as part of another group refers to a monovalent radical aromatic ring system having six to fourteen carbon atoms, i.e., C₆-C₁₄ aryl. Non-limiting Example aryl groups include phenyl (abbreviated as “Ph”), naphthyl, phenanthryl, anthracyl, indenyl, azulenyl, biphenyl, biphenylenyl, and fluorenyl groups. In one example, the aryl group is phenyl or naphthyl. In another example, the aryl group is phenyl. The term aryl also includes aryl groups that are optionally substituted with one, two, or three groups selected from C₁-C₆ alkyl, C₁-C₆ alkoxy, hydroxy, halo, nitro, cyano, and amino.

The term “arylene” refers to a divalent radical corresponding to a monovalent aryl group as defined above. In some particular examples, the arylene corresponds to one of the list of aryl groups provided above.

The term “heteroaryl” as used herein by itself or as part of another group refers to monovalent radical monocyclic aromatic ring systems having five to six ring members, i.e., a 5- to 6-membered heteroaryl, comprising one, two, three, four, or five heteroatoms. Each heteroatom is independently oxygen, sulfur, or nitrogen. In one example, the heteroaryl has three heteroatoms. In another example, the heteroaryl has two heteroatoms. In another example, the heteroaryl has one heteroatom. In another example, the heteroaryl has 5 ring atoms, e.g., furyl, a 5-membered heteroaryl having four carbon atoms and one oxygen atom. In another example, the heteroaryl has 6 ring atoms, e.g., pyridyl, a 6-membered heteroaryl having five carbon atoms and one nitrogen atom. Non-limiting example heteroaryl groups include thienyl, furyl, pyranyl, 2H-pyrrolyl, pyrrolyl, imidazolyl, pyrazolyl, pyridyl, pyrazinyl, pyrimidinyl, pyridazinyl, thiazolyl, isothiazolyl, and isoxazolyl. In one example, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting Example N-oxide is pyridyl N-oxide. The term heteroaryl also includes heteroaryl groups that are optionally substituted with one, two, or three groups selected from C₁-C₆ alkyl, C₁-C₆ alkoxy, hydroxy, halo, nitro, cyano, and amino.

The term “polyalkyleneiminyl” as used herein by itself or as part of another group refers to a linear or branched polymer comprising repeating -(amino)(C₂-C₆ alkyl)- units. In another example, the polyalkyleneiminyl is a linear polymer having the formula:

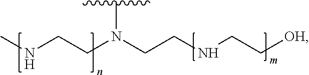

wherein n and m are each independently an integer from 1 to 5000. In another example, the polyalkyleneiminyl is a branched polymer, e.g., a polymer containing the fragment:

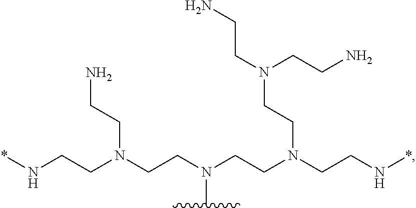

wherein (*) indicates a connection point to additional optionally branched (amino)ethyl repeat units. In another example, the polyalkyleneiminyl has a molecular weight (MW) of from about 1,000 to about 250,000.

The term “polyalkylene glycol” as used herein by itself or as part of another group refers to a linear or branched polymer chain comprising repeating -(hydroxy)(C₂-C₆ alkyl)-units. In some examples, one or more of the C₂-C₆ alkyl groups may be substituted with one or two C₁-C₄ alkyl groups. In another example, the polyalkylene glycol is a linear polymer having the formula:

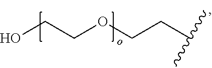

wherein o is an integer from 1 to 5000.

The term “—(C₁-C₆ alkylene)(C₃-C₈ cycloalkylene)(C₁-C₆ alkylene)-” as used herein refers to a C₁-C₆ alkyl which is substituted with a C₃-C₈ cycloalkyl, which is itself substituted with a C₁-C₆ alkyl, which itself contains an additional attachment point. In some examples, —(C₁-C₆ alkylene)(C₃-C₈ cycloalkylene)(C₁-C₆ alkylene)- has the following structure:

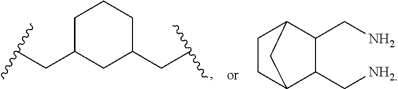 [SMILES: *CC1CCCC(C*)C1 ; NCC1C2CCC(C2)C1CN]

In some examples, the —(C₁-C₆ alkylene)(C₃-C₈ cycloalkylene)(C₁-C₆ alkylene)- is further substituted with an additional group A to form A-(C₁-C₆ alkylene)(C₃-C₈ cycloalkylene)(C₁-C₆ alkylene)-. In some examples, A is amino or —SH. In some examples, A-(C₁-C₆ alkylene)(C₃-C₈ cycloalkylene)(C₁-C₆ alkylene)- has the following structure:

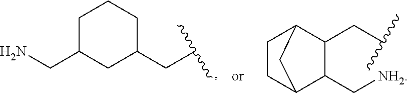 [SMILES: *CC1CCCC(CN)C1 ; *CC1C2CCC(C2)C1CN]

The term “—(C₁-C₆ alkylene)(4- to 7-membered heterocyclylene)(C₁-C₆ alkylene)-” as used herein refers to a C₁-C₆ alkyl which is substituted with a 4- to 7-membered heterocyclyl, which is itself substituted with a C₁-C₆ alkyl, which itself contains an additional attachment point. In one example, —(C₁-C₆ alkylene)(4- to 7-membered heterocyclylene)(C₁-C₆ alkylene)- has the following structure:

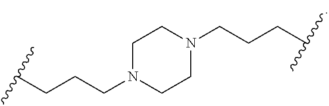 [SMILES: *CCCN1CCN(CCC*)CC1]

In some examples, —(C₁-C₆ alkylene)(4- to 7-membered heterocyclylene)(C₁-C₆ alkylene)- is further substituted with an additional group A to form A-(C₁-C₆ alkylene)(4- to 7-membered heterocyclylene)(C₁-C₆ alkylene)-. In some examples, A is amino or —SH. In one example, A-(C₁-C₆ alkylene)(4- to 7-membered heterocyclylene)(C₁-C₆ alkylene)- has the following structure:

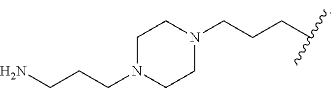 [SMILES: *CCCN1CCN(CCCN)CC1]

The term “—(C₁-C₆ alkylene)(C₆-C₁₄ arylene)(C₁-C₆ alkylene)-” as used herein refers to a C₁-C₆ alkyl which is substituted with a C₆-C₁₄ aryl, which is itself substituted with a C₁-C₆ alkyl, which itself contains an additional attachment point. In one example, —(C₁-C₆ alkylene)(C₆-C₁₄ arylene)(C₁-C₆ alkylene)- has the following structure:

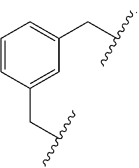 [SMILES: *Cc1cccc(C*)c1]

In some examples, —(C₁-C₆ alkylene)(C₆-C₁₄ arylene)(C₁-C₆ alkylene)- is further substituted with an additional group A to form A-(C₁-C₆ alkylene)(C₆-C₁₄ arylene)(C₁-C₆ alkylene)-. In some examples, A is amino or —SH. In one example, A-(C₁-C₆ alkylene)(C₆-C₁₄ arylene)(C₁-C₆ alkylene)- has the following structure:

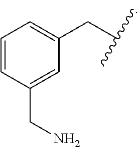 [SMILES: *Cc1cccc(CN)c1]

The term “—(C₃-C₈ cycloalkylene)(C₁-C₆ alkylene)(C₃-C₈ cycloalkylene)-” as used herein refers to a C₃-C₈ cycloalkyl which is substituted with a C₁-C₆ alkyl, which is itself substituted with a C₃-C₈ cycloalkyl, which itself contains an additional attachment point. In one example, —(C₃-C₈ cycloalkylene)(C₁-C₆ alkylene)(C₃-C₈ cycloalkylene)- has the following structure:

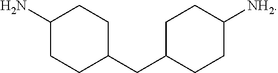 [SMILES: NC1CCC(CC2CCC(N)CC2)CC1]

In some examples, —(C₃-C₈ cycloalkylene)(C₁-C₆ alkylene)(C₃-C₈ cycloalkylene)- is further substituted with an additional group A to form A-(C₃-C₈ cycloalkylene)(C₁-C₆ alkylene)(C₃-C₈ cycloalkylene)-. In some examples, A is amino or —SH. In one example, A-(C₃-C₈ cycloalkylene)(C₁-C₆ alkylene)(C₃-C₈ cycloalkylene)- has the following structure:

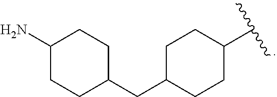 [SMILES: *C1CCC(CC2CCC(N)CC2)CC1]

The term “—(C₁-C₆ alkylene)(4- to 7-membered heterocyclylene)-” refers to a 4- to 7-membered heterocyclyl which is substituted with a C₁-C₆ alkyl, which itself contains an additional attachment point. In some examples, —(C₁-C₆ alkylene)(4- to 7-membered heterocyclyl)- is further substituted with an additional group A to form A-(C₁-C₆ alkylene)(4- to 7-membered heterocyclylene)-. In some examples, A is amino or —SH. In one example, A-(C₁-C₆ alkylene)(4- to 7-membered heterocyclylene)- has the following structure:

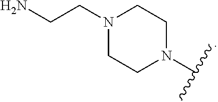 [SMILES: *N1CCN(CCN)CC1]

The term “—(C₁-C₁₂ alkylene)-” refers to a C₁-C₁₂ alkyl which itself contains an additional attachment point. In some examples, it is —(C₁-C₆ alkylene)-, which in one example has the following structure:

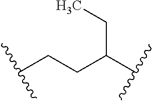 [SMILES: *CCC(*)CC]

In some examples, in the context of R¹—(C₁-C₁₂ alkylene)- is further substituted with an additional group A to form A-(C₁-C₁₂ alkylene)-. In some examples, A is amino or —SH. In some examples, it is A-(C₁-C₆ alkylene)-, which in one example has the following structure:

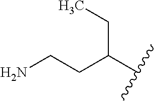 [SMILES: *C(CC)CCN]

### I. COMPOUNDS

The present disclosure provides compounds (also referred to as adducts) which can ligate to luminescent nanostructures. The compounds for example are L-type ligands; that is, Lewis bases which donate two electrons in dative bonding with the nanostructure on ligation. In some examples, when the compounds ligate to the nanostructures, there may be some structural changes to the compound as a result of ligation, e.g., a proton attached to the ligating atom may be lost from the compound. Thus, any reference herein to the compounds or adducts which are ligated to the nanostructures, may also include beyond dative bonding such compounds with structural modifications which result from ligation (but may not be illustrated), e.g., a deprotonated version of the compound.

The present disclosure provides a compound having Formula (I):

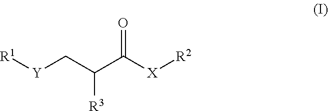

wherein:
Y is —NH— or —S—, and R¹ is selected from the group consisting of:
(i) Z—(C₁-C₆ alkylene)(C₃-C₈ cycloalkylene)(C₁-C₆ alkylene)-,
(ii) Z—(C₁-C₆ alkylene)(4- to 7-membered heterocyclylene)(C₁-C₆ alkylene)-,
(iii) Z—(C₁-C₆ alkylene)(C₆-C₁₄ arylene)(C₁-C₆ alkylene)-,
(iv) Z—(C₃-C₈ cycloalkylene)(C₁-C₆ alkylene)(C₃-C₈ cycloalkylene)-,
(v) polyalkyleneiminyl, and
(vi) Z—(C₁₋₁₂ alkylene)-; or
   Y is absent, R¹ is (vii) Z—(C₁-C₆ alkylene)(4- to 7-membered heterocyclylene)-, and a nitrogen atom in the heterocyclylene provides a point of attachment of R¹ to a carbon in (I) which is β to a carbonyl;
      wherein Z is an amino or —SH; and
      wherein:
R³ is selected from the group consisting of hydrogen and C₁-C₆ alkyl, and R² is selected from the group consisting of:
(i) C₁-C₁₂ alkyl, substituted with zero, one, two, or three groups independently selected from C₁-C₄ alkyl, C₁-C₆ alkoxy, phenoxy, amino, halo, nitro, cyano, and hydroxy;
(ii) C₃-C₈ cycloalkyl, substituted with zero, one, two, or three groups independently selected from C₁-C₄ alkyl, C₁-C₆ alkoxy, amino, halo, nitro, cyano, and hydroxy; and
(iii) polyalkylene glycol, wherein zero, one or more units of the polyalkylene glycol chain is substituted with one or two C₁-C₄ alkyl groups; or
   R² is (iv) C₁₋₁₂alkylene-methacrylate or C₁₋₁₂alkylene-methacrylamide, and R³ is hydrogen, the C₁₋₁₂alkylene is substituted with zero, one or more groups independently selected from C₁-C₄ alkyl, C₁-C₆ alkoxy, phenoxy, amino, halo, nitro, cyano, and hydroxy; and
   wherein X is —NH— or —O—.

In some examples, Z is amino and Y is —NH—. In some examples, R¹ is (amino)(C₁-C₆ alkylene)(C₃-C₈ cycloalkylene)(C₁-C₆ alkylene)-. In some examples, R¹ is:

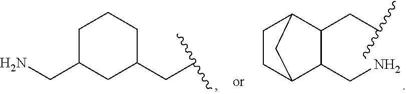 [SMILES: *CC1CCCC(CN)C1 ; *CC1C2CCC(C2)C1CN]

In some examples, R¹ is (amino)(C₁-C₆ alkylene)(4- to 7-membered heterocyclylene)(C₁-C₆ alkylene)-. In some examples, R¹ is:

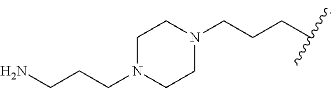 [SMILES: *CCCN1CCN(CCCN)CC1]

In some examples, R¹ is (amino)(C₁-C₆ alkylene)(C₆-C₁₄ arylene)(C₁-C₆ alkylene)-. In some examples, R¹ is:

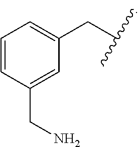 [SMILES: *Cc1cccc(CN)c1]

In some examples, R¹ is (amino)(C₃-C₈ cycloalkylene)(C₁-C₆ alkylene)(C₃-C₈ cycloalkylene)-. In some examples, R¹ is:

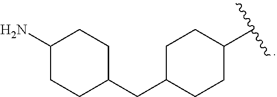 [SMILES: *C1CCC(CC2CCC(N)CC2)CC1]

In some examples, R¹ is (amino)(C₁-C₆ alkylene)(4- to 7-membered heterocyclylene)-, Y is absent, and wherein a nitrogen atom in the heterocyclylene provides the point of attachment of R¹ to the carbon in Formula (I) that is β to the carbonyl group.

In some examples, Y is absent and R¹ is:

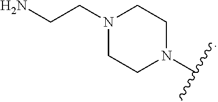 [SMILES: *N1CCN(CCN)CC1]

In some examples, R¹ is polyalkyleneiminyl. In some examples, R¹ is polyethyleneiminyl. In some examples, R¹ is:

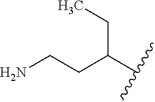 [SMILES: *C(CC)CCN]

In some examples, R² is C₁-C₁₂ alkyl, substituted with zero one, two, or three groups selected from C₁-C₄ alkyl, C₁-C₆ alkoxy, phenoxy, amino, halo, nitro, cyano, and hydroxy. In some examples, R² is selected from the group consisting of:

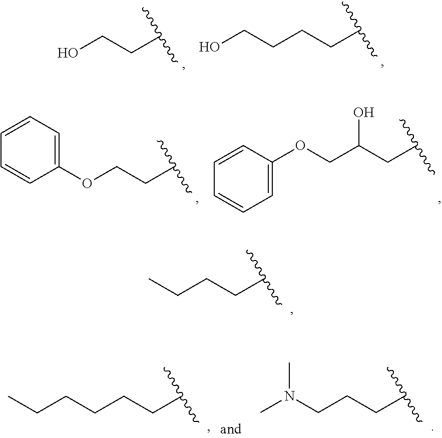

In some examples, R² is C₃-C₈ cycloalkyl, substituted with zero, one, two, or three groups selected from C₁-C₄ alkyl, C₁-C₆ alkoxy, amino, halo, nitro, cyano, and hydroxy. In some examples, R² is:

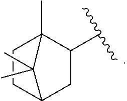 [SMILES: *C1CC2CCC1(C)C2(C)C]

In some examples, R² is polyalkylene glycol. In some examples, R² is polyethylene glycol. In some examples, R² includes a hydroxyl group. In some examples, X is —NH—.

In some examples, X is —O—. In some examples, R³ is hydrogen. In some examples, R³ is methyl. In some examples, R² is C₁₋₁₂alkylene-methacrylate or C₁₋₁₂alkylene-methacrylamide and R³ is hydrogen, wherein the C₁₋₁₂alkylene is substituted with zero, one or more groups independently selected from C₁-C₄ alkyl, C₁-C₆ alkoxy, phenoxy, amino, halo, nitro, cyano, and hydroxy. In some examples, R³ is hydrogen and R² is:

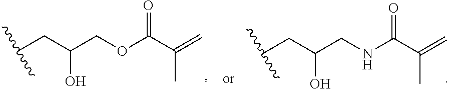 [SMILES: *CC(O)COC(=O)C(=C)C ; *CC(O)CNC(=O)C(=C)C]

In some examples, the compound is selected from the list in Table 1.

TABLE 1

| Compound | |
|---|---|
| No. | Structure |
| 1 | 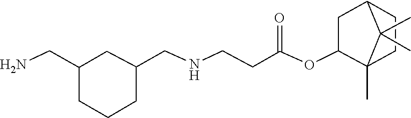 [SMILES: CC1(C)C2CCC1(C)C(OC(=O)CCNCC1CCCC(CN)C1)C2] |
| 2 | 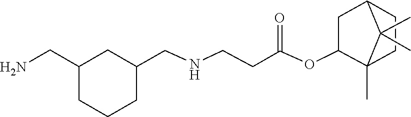 [SMILES: CC1(C)C2CCC1(C)C(OC(=O)CCNCC1CCCC(CN)C1)C2] |
| 3 | 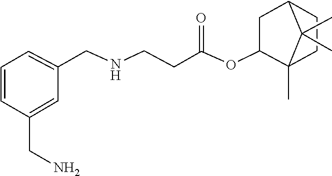 [SMILES: CC1(C)C2CCC1(C)C(OC(=O)CCNCc1cccc(CN)c1)C2] |
| 4 | 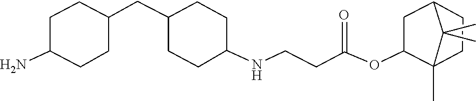 [SMILES: CC1(C)C2CCC1(C)C(OC(=O)CCNC1CCC(CC3CCC(N)CC3)CC1)C2] |
| 5 | 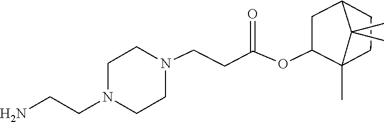 [SMILES: CC1(C)C2CCC1(C)C(OC(=O)CCN1CCN(CCN)CC1)C2] |
| 6 | 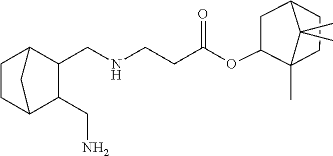 [SMILES: CC1(C)C2CCC1(C)C(OC(=O)CCNCC1C3CCC(C3)C1CN)C2] |
| 7 | 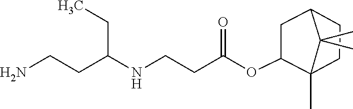 [SMILES: CCC(CCN)NCCC(=O)OC1CC2CCC1(C)C2(C)C] |
| 8 | 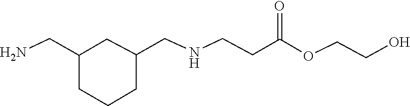 [SMILES: NCC1CCCC(CNCCC(=O)OCCO)C1] |
| 9 | 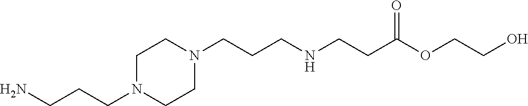 [SMILES: NCCCN1CCN(CCCNCCC(=O)OCCO)CC1] |
| 10 | 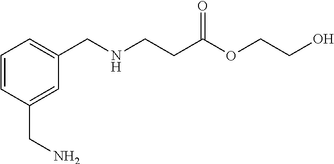 [SMILES: NCc1cccc(CNCCC(=O)OCCO)c1] |
| 11 | 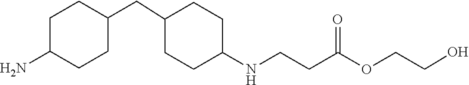 [SMILES: NC1CCC(CC2CCC(NCCC(=O)OCCO)CC2)CC1] |
| 12 | 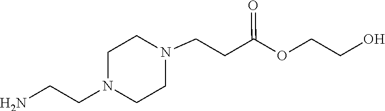 [SMILES: NCCN1CCN(CCC(=O)OCCO)CC1] |
| 13 | 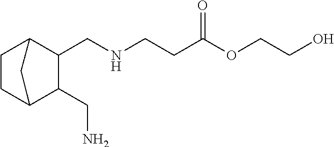 [SMILES: NCC1C2CCC(C2)C1CNCCC(=O)OCCO] |
| 14 | 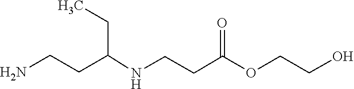 |
| 15 | 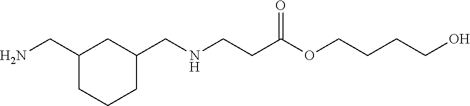 [SMILES: NCC1CCCC(CNCCC(=O)OCCCCO)C1] |
| 16 | 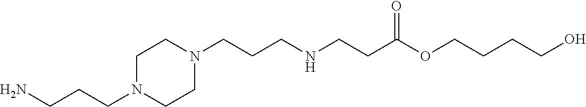 [SMILES: NCCCN1CCN(CCCNCCC(=O)OCCCCO)CC1] |
| 17 | 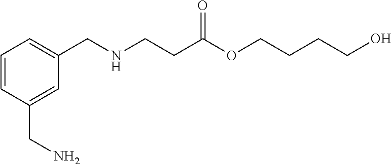 [SMILES: NCc1cccc(CNCCC(=O)OCCCCO)c1] |
| 18 | 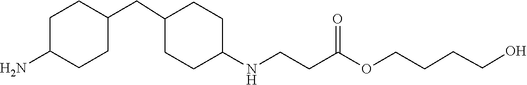 [SMILES: NC1CCC(CC2CCC(NCCC(=O)OCCCCO)CC2)CC1] |
| 19 | 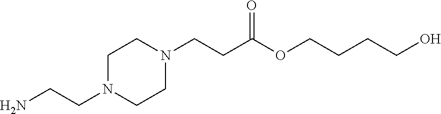 [SMILES: NCCN1CCN(CCC(=O)OCCCCO)CC1] |
| 20 | 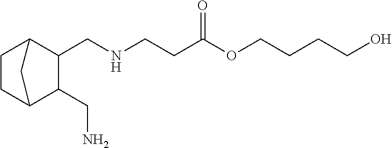 [SMILES: NCC1C2CCC(C2)C1CNCCC(=O)OCCCCO] |
| 21 | 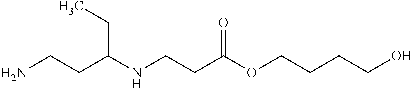 [SMILES: CCC(CCN)NCCC(=O)OCCCCO] |
| 22 | 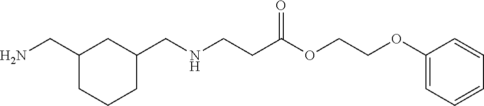 [SMILES: NCC1CCCC(CNCCC(=O)OCCOc2ccccc2)C1] |
| 23 | 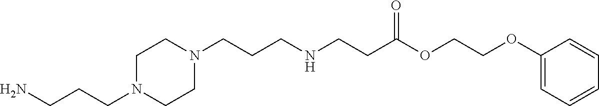 [SMILES: NCCCN1CCN(CCCNCCC(=O)OCCOc2ccccc2)CC1] |
| 24 | 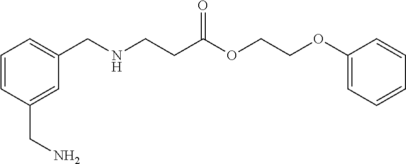 [SMILES: NCc1cccc(CNCCC(=O)OCCOc2ccccc2)c1] |
| 25 | 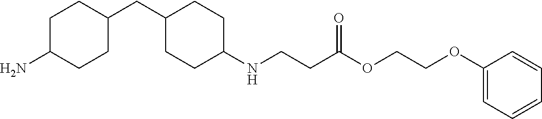 [SMILES: NC1CCC(CC2CCC(NCCC(=O)OCCOc3ccccc3)CC2)CC1] |
| 26 | 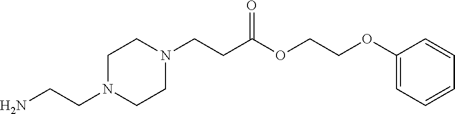 [SMILES: NCCN1CCN(CCC(=O)OCCOc2ccccc2)CC1] |
| 27 | 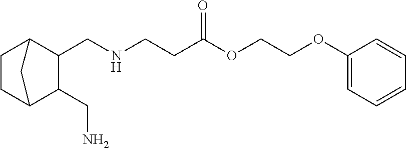 [SMILES: NCC1C2CCC(C2)C1CNCCC(=O)OCCOc1ccccc1] |
| 28 | 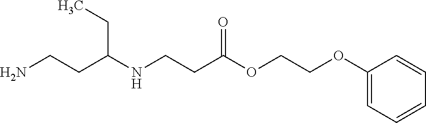 [SMILES: CCC(CCN)NCCC(=O)OCCOc1ccccc1] |
| 29 | 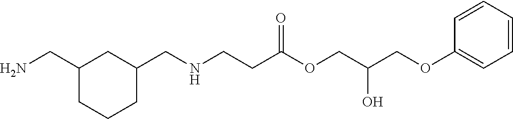 [SMILES: NCC1CCCC(CNCCC(=O)OCC(O)COc2ccccc2)C1] |
| 30 | 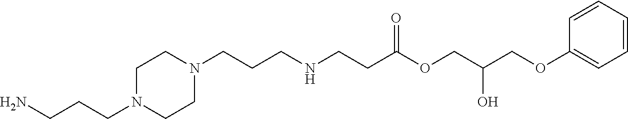 [SMILES: NCCCN1CCN(CCCNCCC(=O)OCC(O)COc2ccccc2)CC1] |
| 31 | 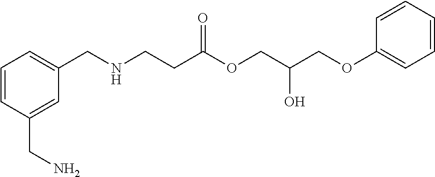 [SMILES: NCc1cccc(CNCCC(=O)OCC(O)COc2ccccc2)c1] |
| 32 | 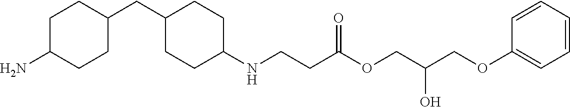 [SMILES: NC1CCC(CC2CCC(NCCC(=O)OCC(O)COc3ccccc3)CC2)CC1] |
| 33 | 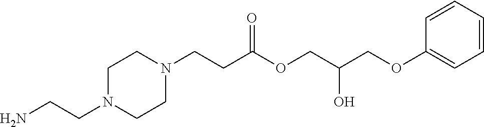 [SMILES: NCCN1CCN(CCC(=O)OCC(O)COc2ccccc2)CC1] |
| 34 | 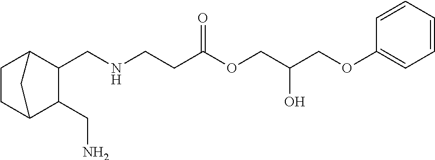 [SMILES: NCC1C2CCC(C2)C1CNCCC(=O)OCC(O)COc1ccccc1] |
| 35 | 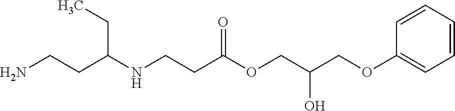 [SMILES: CCC(CCN)NCCC(=O)OCC(O)COc1ccccc1] |
| 36 | 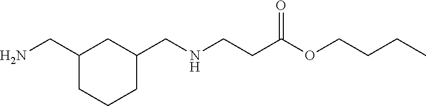 [SMILES: CCCCOC(=O)CCNCC1CCCC(CN)C1] |
| 37 | 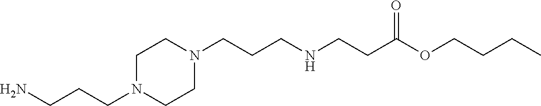 [SMILES: CCCCOC(=O)CCNCCCN1CCN(CCCN)CC1] |
| 38 | 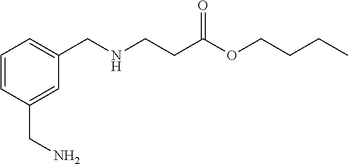 [SMILES: CCCCOC(=O)CCNCc1cccc(CN)c1] |
| 39 | 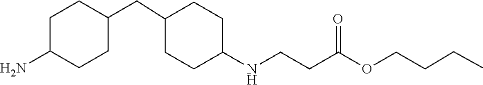 [SMILES: CCCCOC(=O)CCNC1CCC(CC2CCC(N)CC2)CC1] |
| 40 | 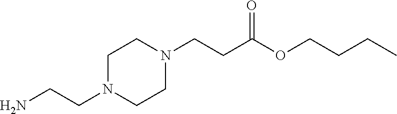 [SMILES: CCCCOC(=O)CCN1CCN(CCN)CC1] |
| 41 | 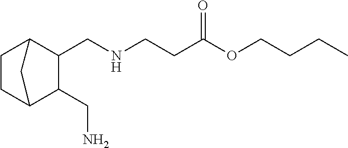 [SMILES: CCCCOC(=O)CCNCC1C2CCC(C2)C1CN] |
| 42 | 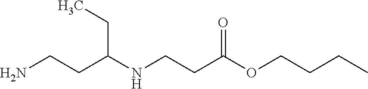 [SMILES: CCCCOC(=O)CCNC(CC)CCN] |
| 43 | 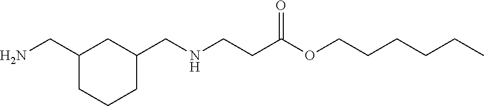 [SMILES: CCCCCCOC(=O)CCNCC1CCCC(CN)C1] |
| 44 | 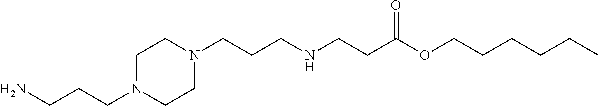 [SMILES: CCCCCCOC(=O)CCNCCCN1CCN(CCCN)CC1] |
| 45 | 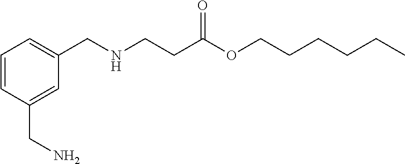 [SMILES: CCCCCCOC(=O)CCNCc1cccc(CN)c1] |
| 46 | 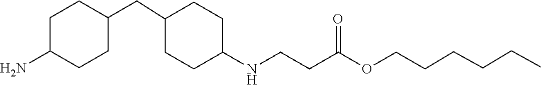 [SMILES: CCCCCCOC(=O)CCNC1CCC(CC2CCC(N)CC2)CC1] |
| 47 | 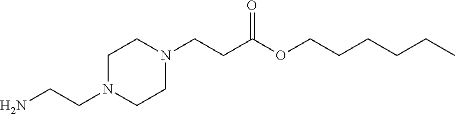 [SMILES: CCCCCCOC(=O)CCN1CCN(CCN)CC1] |
| 48 | 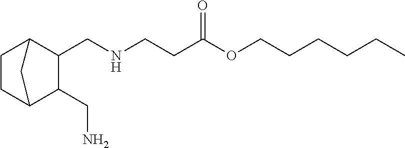 [SMILES: CCCCCCOC(=O)CCNCC1C2CCC(C2)C1CN] |
| 49 | 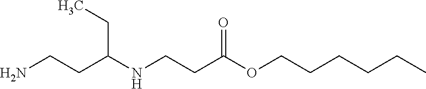 [SMILES: CCCCCCOC(=O)CCNC(CC)CCN] |
| 50 | 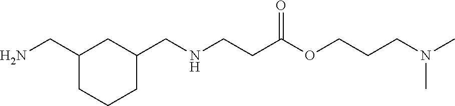 [SMILES: CN(C)CCCOC(=O)CCNCC1CCCC(CN)C1] |
| 51 | 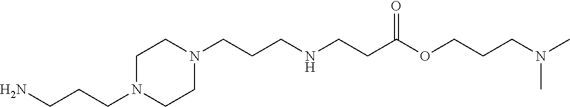 [SMILES: CN(C)CCCOC(=O)CCNCCCN1CCN(CCCN)CC1] |
| 52 | 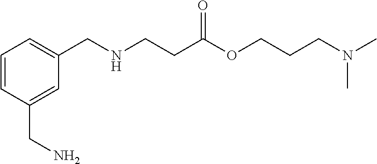 [SMILES: CN(C)CCCOC(=O)CCNCc1cccc(CN)c1] |
| 53 | 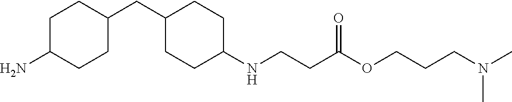 [SMILES: CN(C)CCCOC(=O)CCNC1CCC(CC2CCC(N)CC2)CC1] |
| 54 | 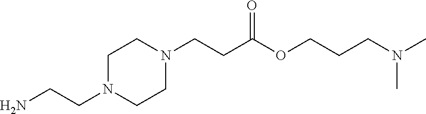 [SMILES: CN(C)CCCOC(=O)CCN1CCN(CCN)CC1] |
| 55 | 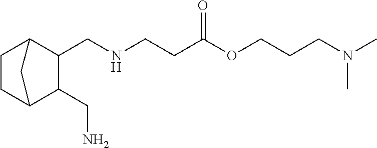 [SMILES: CN(C)CCCOC(=O)CCNCC1C2CCC(C2)C1CN] |
| 56 | 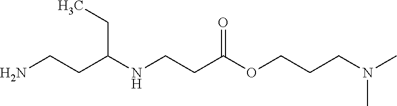 [SMILES: CCC(CCN)NCCC(=O)OCCCN(C)C] |
| 57 | 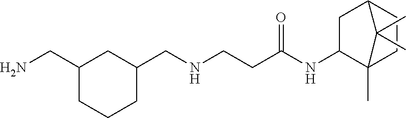 [SMILES: CC1(C)C2CCC1(C)C(NC(=O)CCNCC1CCCC(CN)C1)C2] |
| 58 | 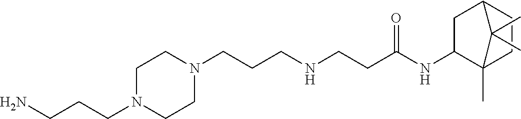 [SMILES: CC1(C)C2CCC1(C)C(NC(=O)CCNCCCN1CCN(CCCN)CC1)C2] |
| 59 | 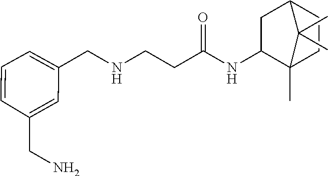 [SMILES: CC1(C)C2CCC1(C)C(NC(=O)CCNCc1cccc(CN)c1)C2] |
| 60 | 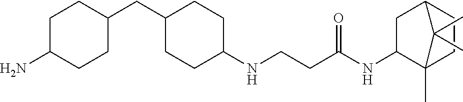 [SMILES: CC1(C)C2CCC1(C)C(NC(=O)CCNC1CCC(CC3CCC(N)CC3)CC1)C2] |
| 61 | 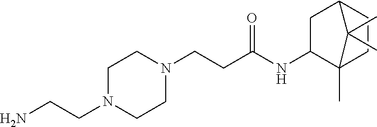 [SMILES: CC1(C)C2CCC1(C)C(NC(=O)CCN1CCN(CCN)CC1)C2] |
| 62 | 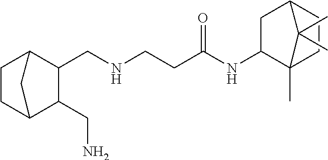 [SMILES: CC1(C)C2CCC1(C)C(NC(=O)CCNCC1C3CCC(C3)C1CN)C2] |
| 63 | 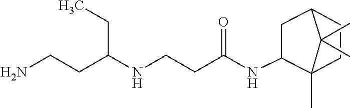 [SMILES: CCC(CCN)NCCC(=O)NC1CC2CCC1(C)C2(C)C] |
| 64 | 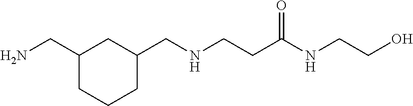 [SMILES: NCC1CCCC(CNCCC(=O)NCCO)C1] |
| 65 | 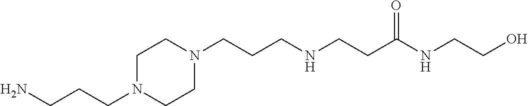 [SMILES: NCCCN1CCN(CCCNCCC(=O)NCCO)CC1] |
| 66 | 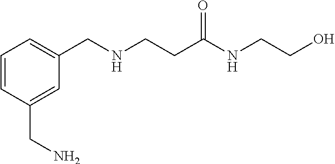 [SMILES: NCc1cccc(CNCCC(=O)NCCO)c1] |
| 67 | 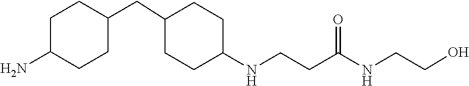 [SMILES: NC1CCC(CC2CCC(NCCC(=O)NCCO)CC2)CC1] |
| 68 | 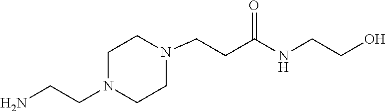 [SMILES: NCCN1CCN(CCC(=O)NCCO)CC1] |
| 69 | 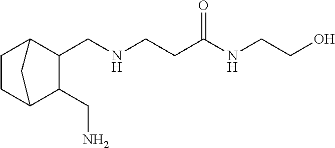 [SMILES: NCC1C2CCC(C2)C1CNCCC(=O)NCCO] |
| 70 | 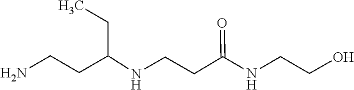 |
| 71 | 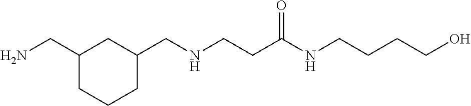 [SMILES: NCC1CCCC(CNCCC(=O)NCCCCO)C1] |
| 72 | 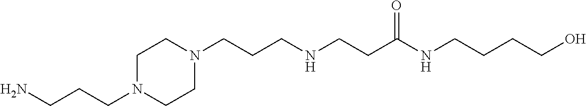 [SMILES: NCCCN1CCN(CCCNCCC(=O)NCCCCO)CC1] |
| 73 | 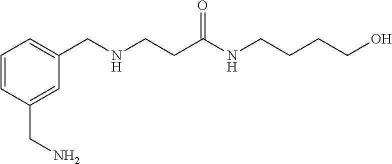 [SMILES: NCc1cccc(CNCCC(=O)NCCCCO)c1] |
| 74 | 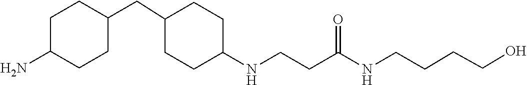 [SMILES: NC1CCC(CC2CCC(NCCC(=O)NCCCCO)CC2)CC1] |
| 75 | 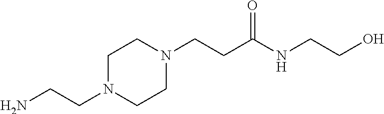 [SMILES: NCCN1CCN(CCC(=O)NCCO)CC1] |
| 76 | 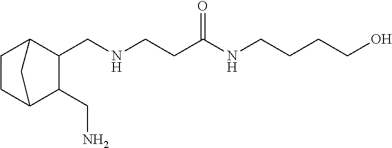 [SMILES: NCC1C2CCC(C2)C1CNCCC(=O)NCCCCO] |
| 77 | 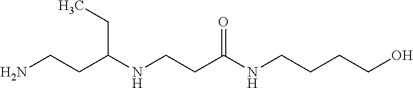 [SMILES: CCC(CCN)NCCC(=O)NCCCCO] |
| 78 | 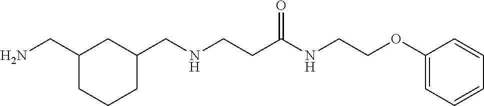 [SMILES: NCC1CCCC(CNCCC(=O)NCCOc2ccccc2)C1] |
| 79 | 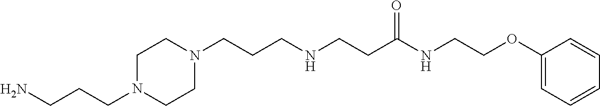 [SMILES: NCCCN1CCN(CCCNCCC(=O)NCCOc2ccccc2)CC1] |
| 80 | 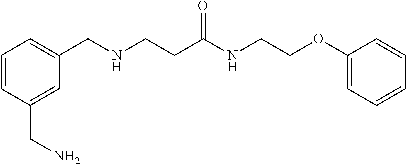 [SMILES: NCc1cccc(CNCCC(=O)NCCOc2ccccc2)c1] |
| 81 | 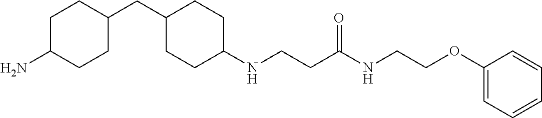 [SMILES: NC1CCC(CC2CCC(NCCC(=O)NCCOc3ccccc3)CC2)CC1] |
| 82 | 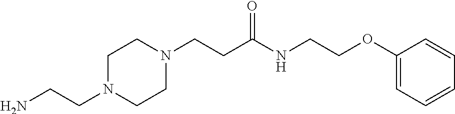 [SMILES: NCCN1CCN(CCC(=O)NCCOc2ccccc2)CC1] |
| 83 | 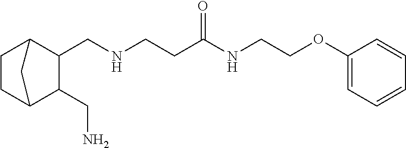 [SMILES: NCC1C2CCC(C2)C1CNCCC(=O)NCCOc1ccccc1] |
| 84 | 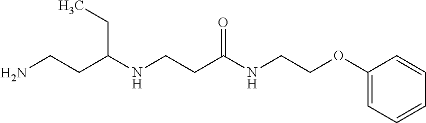 [SMILES: CCC(CCN)NCCC(=O)NCCOc1ccccc1] |
| 85 | 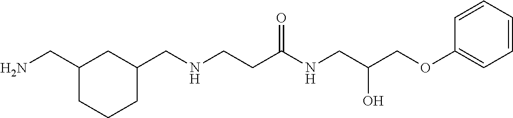 [SMILES: NCC1CCCC(CNCCC(=O)NCC(O)COc2ccccc2)C1] |
| 86 | 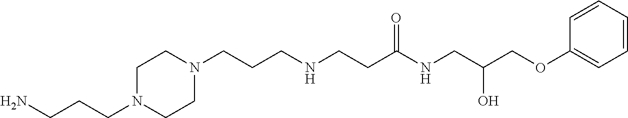 [SMILES: NCCCN1CCN(CCCNCCC(=O)NCC(O)COc2ccccc2)CC1] |
| 87 | 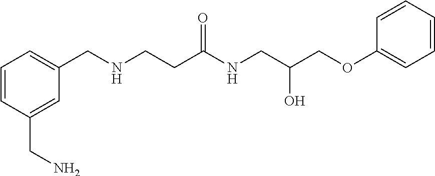 [SMILES: NCc1cccc(CNCCC(=O)NCC(O)COc2ccccc2)c1] |
| 88 | 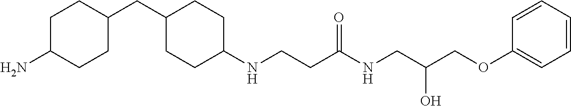 [SMILES: NC1CCC(CC2CCC(NCCC(=O)NCC(O)COc3ccccc3)CC2)CC1] |
| 89 | 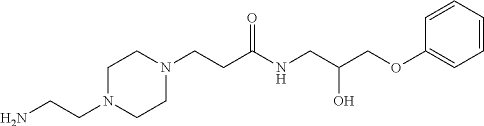 [SMILES: NCCN1CCN(CCC(=O)NCC(O)COc2ccccc2)CC1] |
| 90 | 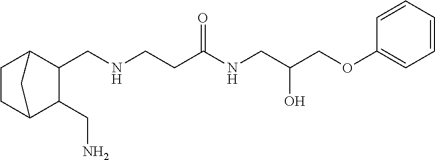 [SMILES: NCC1C2CCC(C2)C1CNCCC(=O)NCC(O)COc1ccccc1] |
| 91 | 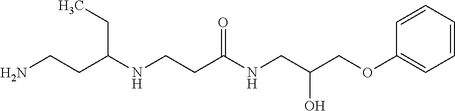 [SMILES: CCC(CCN)NCCC(=O)NCC(O)COc1ccccc1] |
| 92 | 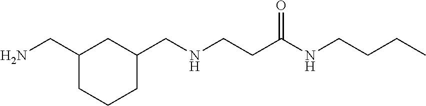 [SMILES: CCCCNC(=O)CCNCC1CCCC(CN)C1] |
| 93 | 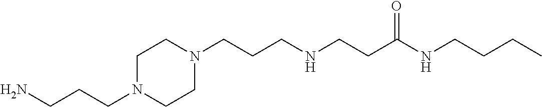 [SMILES: CCCCNC(=O)CCNCCCN1CCN(CCCN)CC1] |
| 94 | 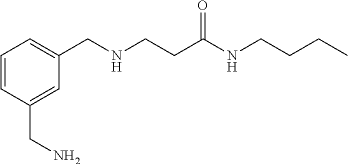 [SMILES: CCCCNC(=O)CCNCc1cccc(CN)c1] |
| 95 | 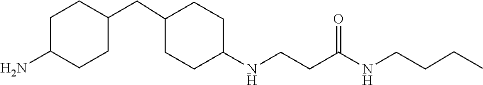 [SMILES: CCCCNC(=O)CCNC1CCC(CC2CCC(N)CC2)CC1] |
| 96 | 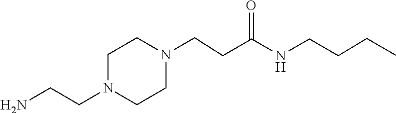 [SMILES: CCCCNC(=O)CCN1CCN(CCN)CC1] |
| 97 | 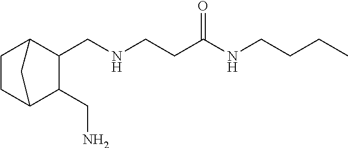 [SMILES: CCCCNC(=O)CCNCC1C2CCC(C2)C1CN] |
| 98 | 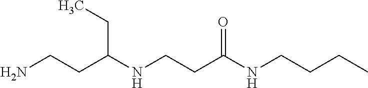 [SMILES: CCCCNC(=O)CCNC(CC)CCN] |
| 99 | 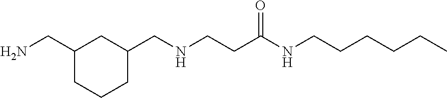 [SMILES: CCCCCCNC(=O)CCNCC1CCCC(CN)C1] |
| 100 | 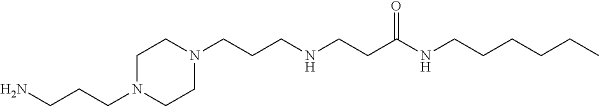 [SMILES: CCCCCCNC(=O)CCNCCCN1CCN(CCCN)CC1] |
| 101 | 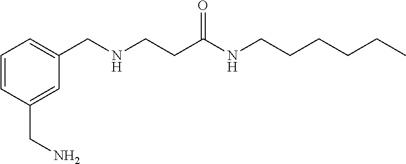 [SMILES: CCCCCCNC(=O)CCNCc1cccc(CN)c1] |
| 102 | 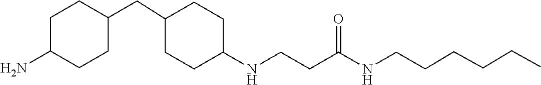 [SMILES: CCCCCCNC(=O)CCNC1CCC(CC2CCC(N)CC2)CC1] |
| 103 | 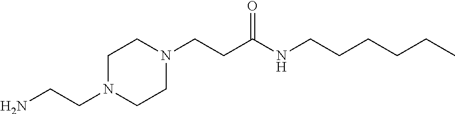 [SMILES: CCCCCCNC(=O)CCN1CCN(CCN)CC1] |
| 104 | 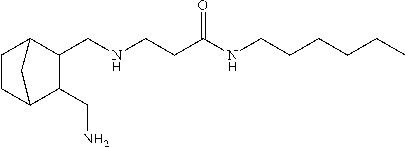 [SMILES: CCCCCCNC(=O)CCNCC1C2CCC(C2)C1CN] |
| 105 | 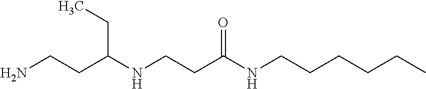 [SMILES: CCCCCCNC(=O)CCNC(CC)CCN] |
| 106 | 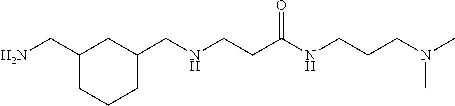 [SMILES: CN(C)CCCNC(=O)CCNCC1CCCC(CN)C1] |
| 107 | 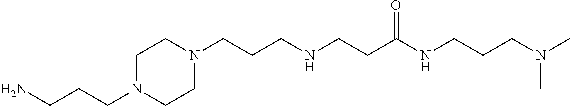 [SMILES: CN(C)CCCNC(=O)CCNCCCN1CCN(CCCN)CC1] |
| 108 | 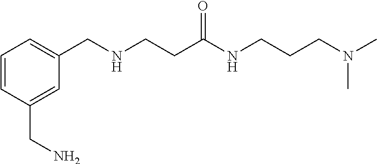 [SMILES: CN(C)CCCNC(=O)CCNCc1cccc(CN)c1] |
| 109 | 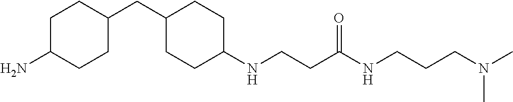 [SMILES: CN(C)CCCNC(=O)CCNC1CCC(CC2CCC(N)CC2)CC1] |
| 110 | 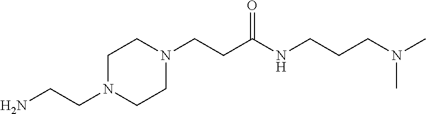 [SMILES: CN(C)CCCNC(=O)CCN1CCN(CCN)CC1] |
| 111 | 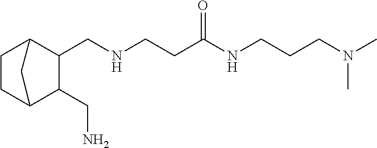 [SMILES: CN(C)CCCNC(=O)CCNCC1C2CCC(C2)C1CN] |
| 112 | 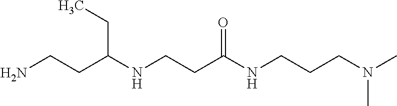 [SMILES: CCC(CCN)NCCC(=O)NCCCN(C)C] |
| 113 | 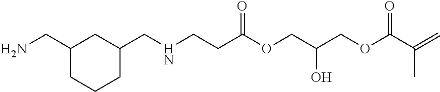 [SMILES: C=C(C)C(=O)OCC(O)COC(=O)CCNCC1CCCC(CN)C1] |
| 114 | 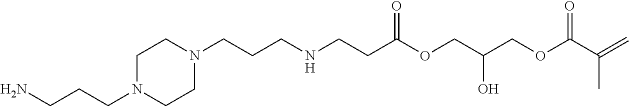 [SMILES: C=C(C)C(=O)OCC(O)COC(=O)CCNCCCN1CCN(CCCN)CC1] |
| 115 | 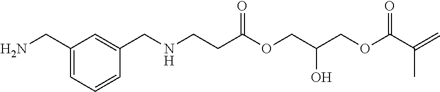 [SMILES: C=C(C)C(=O)OCC(O)COC(=O)CCNCc1cccc(CN)c1] |
| 116 | 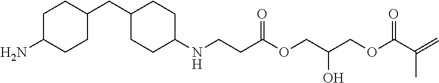 [SMILES: C=C(C)C(=O)OCC(O)COC(=O)CCNC1CCC(CC2CCC(N)CC2)CC1] |
| 117 | 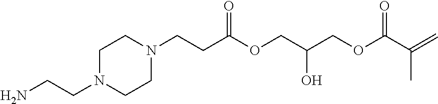 [SMILES: C=C(C)C(=O)OCC(O)COC(=O)CCN1CCN(CCN)CC1] |
| 118 | 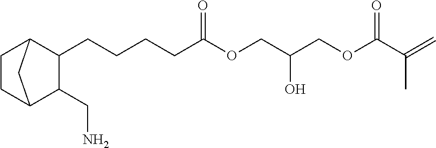 [SMILES: C=C(C)C(=O)OCC(O)COC(=O)CCCCC1C2CCC(C2)C1CN] |
| 119 | 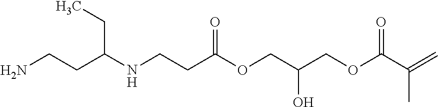 [SMILES: C=C(C)C(=O)OCC(O)COC(=O)CCNC(CC)CCN] |
| 120 | 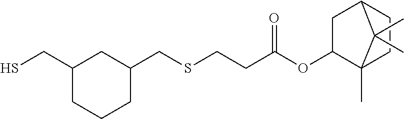 [SMILES: CC1(C)C2CCC1(C)C(OC(=O)CCSCC1CCCC(CS)C1)C2] |
| 121 | 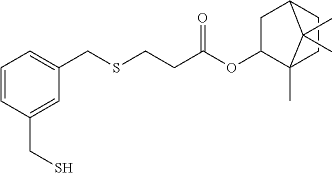 [SMILES: CC1(C)C2CCC1(C)C(OC(=O)CCSCc1cccc(CS)c1)C2] |
| 122 | 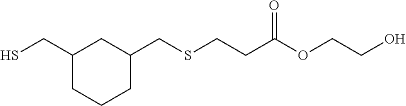 [SMILES: O=C(CCSCC1CCCC(CS)C1)OCCO] |
| 123 | 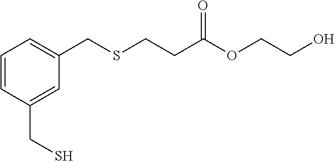 [SMILES: O=C(CCSCc1cccc(CS)c1)OCCO] |
| 124 | 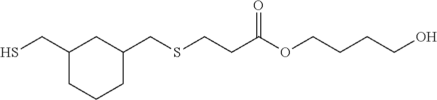 [SMILES: O=C(CCSCC1CCCC(CS)C1)OCCCCO] |
| 125 | 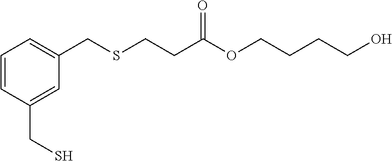 [SMILES: O=C(CCSCc1cccc(CS)c1)OCCCCO] |
| 126 | 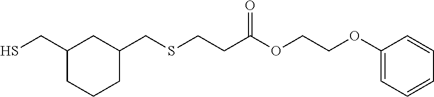 [SMILES: O=C(CCSCC1CCCC(CS)C1)OCCOc1ccccc1] |
| 127 | 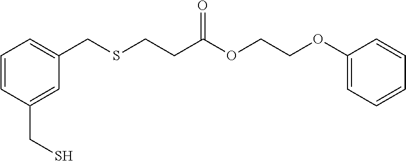 [SMILES: O=C(CCSCc1cccc(CS)c1)OCCOc1ccccc1] |
| 128 | 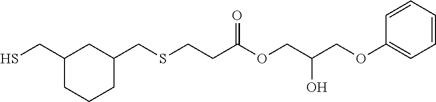 [SMILES: O=C(CCSCC1CCCC(CS)C1)OCC(O)COc1ccccc1] |
| 129 | 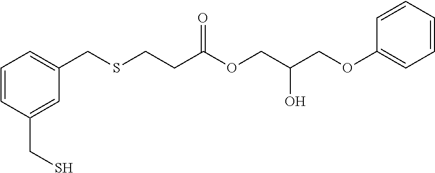 [SMILES: O=C(CCSCc1cccc(CS)c1)OCC(O)COc1ccccc1] |
| 130 | 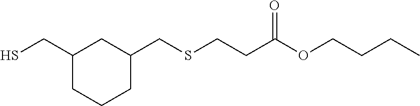 [SMILES: CCCCOC(=O)CCSCC1CCCC(CS)C1] |
| 131 | 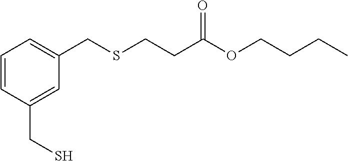 [SMILES: CCCCOC(=O)CCSCc1cccc(CS)c1] |
| 132 | 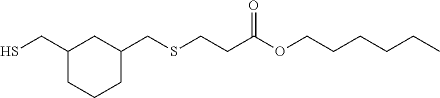 [SMILES: CCCCCCOC(=O)CCSCC1CCCC(CS)C1] |
| 133 | 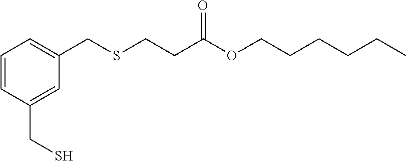 [SMILES: CCCCCCOC(=O)CCSCc1cccc(CS)c1] |
| 134 | 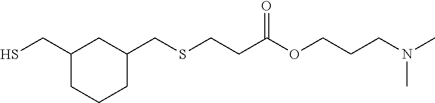 [SMILES: CN(C)CCCOC(=O)CCSCC1CCCC(CS)C1] |
| 135 | 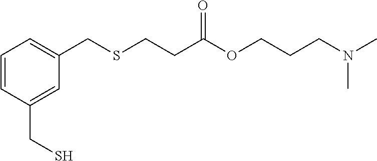 [SMILES: CN(C)CCCOC(=O)CCSCc1cccc(CS)c1] |
| 136 | 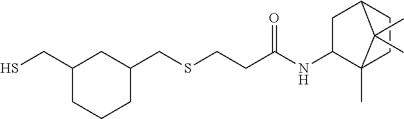 [SMILES: CC1(C)C2CCC1(C)C(NC(=O)CCSCC1CCCC(CS)C1)C2] |
| 137 | 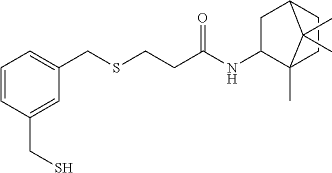 [SMILES: CC1(C)C2CCC1(C)C(NC(=O)CCSCc1cccc(CS)c1)C2] |
| 138 | 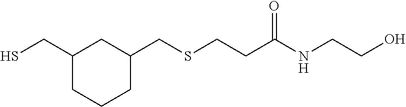 [SMILES: O=C(CCSCC1CCCC(CS)C1)NCCO] |
| 139 | 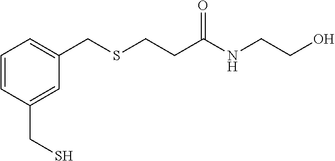 [SMILES: O=C(CCSCc1cccc(CS)c1)NCCO] |
| 140 | 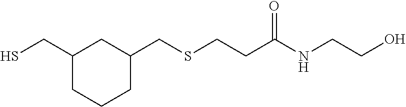 [SMILES: O=C(CCSCC1CCCC(CS)C1)NCCO] |
| 141 | 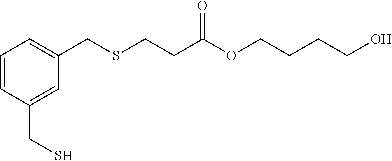 [SMILES: O=C(CCSCc1cccc(CS)c1)OCCCCO] |
| 142 | 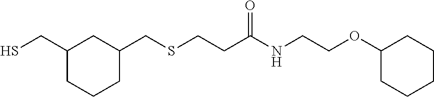 [SMILES: O=C(CCSCC1CCCC(CS)C1)NCCOC1CCCCC1] |
| 143 | 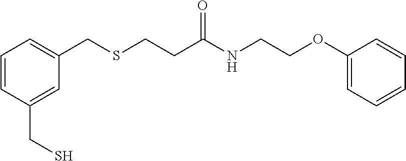 [SMILES: O=C(CCSCc1cccc(CS)c1)NCCOc1ccccc1] |
| 144 | 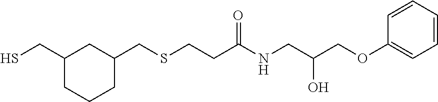 [SMILES: O=C(CCSCC1CCCC(CS)C1)NCC(O)COc1ccccc1] |
| 145 | 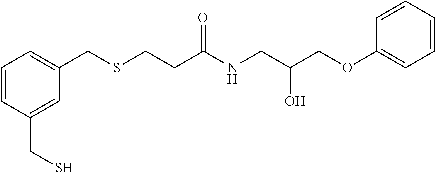 [SMILES: O=C(CCSCc1cccc(CS)c1)NCC(O)COc1ccccc1] |
| 146 | 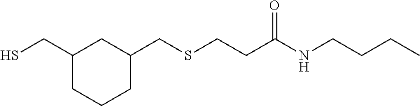 [SMILES: CCCCNC(=O)CCSCC1CCCC(CS)C1] |
| 147 | 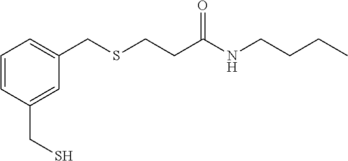 [SMILES: CCCCNC(=O)CCSCc1cccc(CS)c1] |
| 148 | 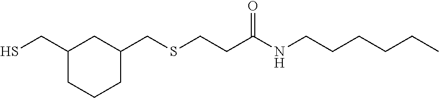 [SMILES: CCCCCCNC(=O)CCSCC1CCCC(CS)C1] |
| 149 | 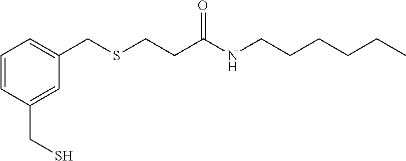 [SMILES: CCCCCCNC(=O)CCSCc1cccc(CS)c1] |
| 150 | 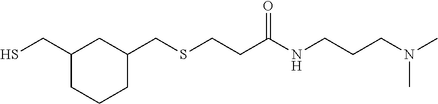 [SMILES: CN(C)CCCNC(=O)CCSCC1CCCC(CS)C1] |
| 151 | 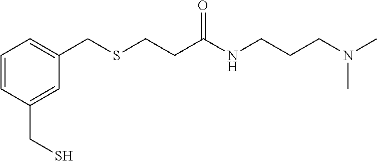 [SMILES: CN(C)CCCNC(=O)CCSCc1cccc(CS)c1] |
| 152 | 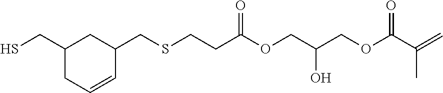 [SMILES: C=C(C)C(=O)OCC(O)COC(=O)CCSCC1C=CCC(CS)C1] |
| 153 | 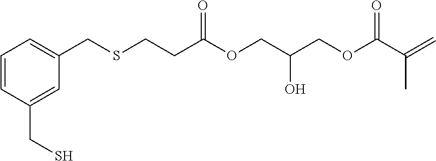 [SMILES: C=C(C)C(=O)OCC(O)COC(=O)CCSCc1cccc(CS)c1] |
| 154 | 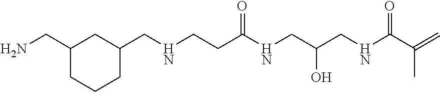 [SMILES: C=C(C)C(=O)NCC(O)CNC(=O)CCNCC1CCCC(CN)C1] |
| 155 | 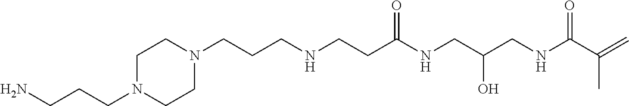 [SMILES: C=C(C)C(=O)NCC(O)CNC(=O)CCNCCCN1CCN(CCCN)CC1] |
| 156 | 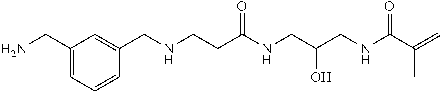 [SMILES: C=C(C)C(=O)NCC(O)CNC(=O)CCNCc1cccc(CN)c1] |
| 157 | 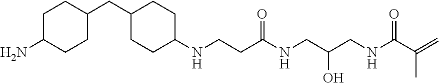 [SMILES: C=C(C)C(=O)NCC(O)CNC(=O)CCNC1CCC(CC2CCC(N)CC2)CC1] |
| 158 | 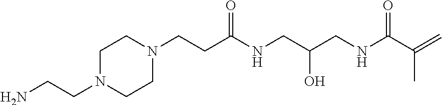 [SMILES: C=C(C)C(=O)NCC(O)CNC(=O)CCN1CCN(CCN)CC1] |
| 159 | 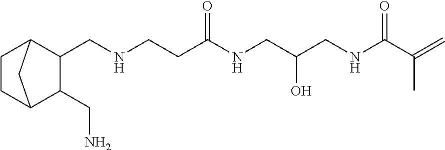 [SMILES: C=C(C)C(=O)NCC(O)CNC(=O)CCNCC1C2CCC(C2)C1CN] |
| 160 | 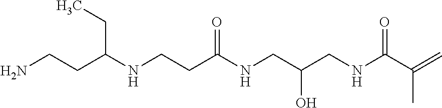 [SMILES: C=C(C)C(=O)NCC(O)CNC(=O)CCNC(CC)CCN] |
| 161 | 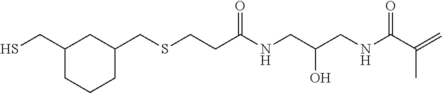 [SMILES: C=C(C)C(=O)NCC(O)CNC(=O)CCSCC1CCCC(CS)C1] |
| 162 | 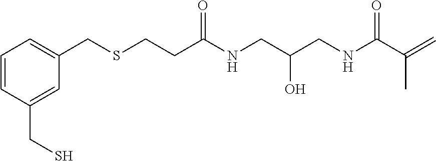 [SMILES: C=C(C)C(=O)NCC(O)CNC(=O)CCSCc1cccc(CS)c1] |

### II. PARTICLES

The luminescent nanostructures for use in examples herein can be produced from any suitable material, suitably an inorganic material, and more suitably an inorganic conductive or semiconductive material. Suitable semiconductor materials include any type of semiconductor, including Group II-VI, Group III-V, Group IV-VI, and Group IV semiconductors. Suitable semiconductor materials include, but are not limited to, Si, Ge, Sn, Se, Te, B, C (including diamond), P, BN, BP, BAs, AlN, AlP, AlAs, AlSb, AgInS, AgGaS, AgInGaS, GaN, GaP, GaAs, GaSb, InN, InP, InAs, InSb, ZnO, ZnS, ZnSe, ZnTe, CdS, CdSe, CdSeZn, CdTe, HgS, HgSe, HgTe, BeS, BeSe, BeTe, MgS, MgSe, GeS, GeSe, GeTe, SnS, SnSe, SnTe, PbO, PbS, PbSe, PbTe, CuF, CuCl, CuBr, CuI, Si₃N₄, Ge₃N₄, Al₂O₃, Al₂CO, and combinations thereof.

In some examples, the core is a Group II-VI nanocrystal selected from the group consisting of ZnO, ZnSe, ZnS, ZnTe, CdO, CdSe, CdS, CdTe, HgO, HgSe, HgS, and HgTe.

Although Group II-VI nanostructures such as CdSe and CdS quantum dots can exhibit desirable luminescence behavior, issues such as the toxicity of cadmium limit the applications for which such nanostructures can be used. Less toxic alternatives with favorable luminescence properties are thus highly desirable. Group III-V nanostructures in general and InP-based nanostructures in particular, offer the best known substitute for cadmium-based materials due to their compatible emission range. AgInGaS (AIGS) nanostructures are also a less toxic alternative.

In some examples, the nanostructures are free from cadmium. As used herein, the term “free of cadmium” is intended that the nanostructures contain less than 100 ppm by weight of cadmium. The Restriction of Hazardous Substances (RoHS) compliance definition requires that there must be no more than 0.01% (100 ppm) by weight of cadmium in the raw homogeneous precursor materials. The cadmium level in the Cd-free nanostructures is limited by the trace metal concentration in the precursor materials. The trace metal (including cadmium) concentration in the precursor materials for the Cd-free nanostructures, can be measured by inductively coupled plasma mass spectroscopy (ICP-MS) analysis, and are on the parts per billion (ppb) level. In some examples, nanostructures that are “free of cadmium” contain less than about 50 ppm, less than about 20 ppm, less than about 10 ppm, or less than about 1 ppm of cadmium.

Example materials for preparing shells for a core-shell nanostructure include, but are not limited to, Si, Ge, Sn, Se, Te, B, C (including diamond), P, Co, Au, BN, BP, BAs, AlN, AlP, AlAs, AlSb, GaN, GaP, GaAs, GaS, GaSb, InN, InP, InAs, InSb, GaSb, ZnO, ZnS, ZnSe, ZnTe, CdS, CdSe, CdSeZn, CdTe, HgS, HgSe, HgTe, BeS, BeSe, BeTe, MgS, MgSe, GeS, GeSe, GeTe, SnS, SnSe, SnTe, PbO, PbS, PbSe, PbTe, CuF, CuCl, CuBr, CuI, Si₃N₄, Ge₃N₄, Al₂O₃, Al₂CO, and combinations thereof.

Example core/shell luminescent nanostructures include, but are not limited to, (represented as core/shell or core/shell/shell) CdSe/ZnSe, InP/ZnSe, InP/ZnSe/ZnS, and AgInGaS/GaS.

In some examples, the luminescent nanostructure is synthesized in the presence of at least one compound which forms a ligand on the surface of the structure. Following synthesis, any ligand on the surface of the nanostructures can be exchanged for a different ligand with other desirable properties. Ligands can, e.g., enhance the miscibility of nanostructures in solvents or polymers (e.g., allowing the nanostructures to be distributed throughout a composition such that the nanostructures do not aggregate together), increase quantum yield of nanostructures (e.g., protect the nanostructure from deterioration on exposure to environmental conditions), and/or preserve nanostructure luminescence (e.g., when the nanostructures are incorporated into the UV-cured monomers).

Ligands suitable for use during synthesis of luminescent nanostructures are known by those of skill in the art. In some examples, the ligand is a fatty acid selected from the group consisting of lauric acid, caproic acid, myristic acid, palmitic acid, stearic acid, and oleic acid. In some examples, the ligand is an organic phosphine or an organic phosphine oxide selected from trioctylphosphine oxide (TOPO), trioctylphosphine (TOP), diphenylphosphine (DPP), triphenylphosphine oxide, and tributylphosphine oxide. In some examples, the ligand is an amine selected from the group consisting of dodecylamine, oleylamine, hexadecylamine, dioctylamine, and octadecylamine. In some examples, the ligand is oleic acid.

In some examples, the luminescent nanostructures comprise Ag, In, Ga, and S (AIGS). In some examples, the nanostructures have a luminescence-wavelength peak in the range of 480-545 nanometres. In some examples, at least about 80% of the luminescence is band-edge emission. The quantum yield of the luminescent nanostructures may be greater than at least one of 0.7, 0.8, 0.9, 0.95 or 0.99.

In some examples herein, there is provided a particle comprising a luminescent nanostructure; and a ligand; the ligand comprising at least one compound of Formula (I).

In some examples, the particle comprises a second ligand bound to the nanostructure, wherein the second ligand is a compound according to Formula (I) and is different from the first ligand.

In some examples, the luminescent nanostructure comprises Si, Ge, Sn, Se, Te, B, C, P, BN, BP, BAs, AlN, AlP, AlAs, AlSb, AgInS, AgGaS, AgInGaS, GaN, GaP, GaAs, GaSb, InN, InP, InAs, InSb, ZnO, ZnS, ZnSe, ZnTe, CdS, CdSe, CdSeZn, CdTe, HgS, HgSe, HgTe, BeS, BeSe, BeTe, MgS, MgSe, GeS, GeSe, GeTe, SnS, SnSe, SnTe, PbO, PbS, PbSe, PbTe, CuF, CuCi, CuBr, CuI, Si₃N₄, Ge₃N₄, Al₂O₃, Al₂CO, or a combination thereof. In some examples, the luminescent nanostructure has a core-shell structure. In some such examples, the core comprises AgInGaS and the shell comprises GaS.

Particles according to some examples may be made by mixing two of: a Michael-acceptor compound, wherein the Michael-acceptor compound comprises an acrylate, methacrylate, acrylamide or methacrylamide group; a Michael-donor compound, wherein the Michael-donor compound is selected from a di-amine, a di-thiol or a compound having an amine group and a thiol group, and wherein a Michael reaction between the Michael-donor and Michael-acceptor compounds forms an adduct; and a population of luminescent nanostructures, to form a mixture; and adding the third of (i) to (iii) to the mixture, wherein a Michael reaction between the Michael-donor and Michael-acceptor compounds forms an adduct, and wherein the particles comprise a luminescent nanostructure and a ligand, wherein the ligand comprises the adduct.

In some examples, the particles are prepared by admixing an ex situ synthesized ligand with a population of nanostructures. In some examples, the particles are prepared by admixing precursors for a ligand, e.g., a Michael donor and a Michael acceptor, with a population of luminescent nanostructures to form the ligand in situ. The precursors may be added to the population of nanostructures in any order.

Thus, in some examples (i) and (ii) are mixed initially to form the adduct and (iii) is subsequently added to the mixture. This may be referred to herein and ex-situ formation of the adduct or ligand. In other examples, (i) or (ii) is mixed with (iii) initially, and then the other of (i) and (ii) is added to the mixture. The ligand/adduct forms in the presence of the luminescent nanostructure and this may be referred to herein as in-situ ligand/adduct formation.

Accordingly, this disclosure provides different methods of making a population of particles, as summarized in aspects P37-P39 hereinafter. Each method offers a different advantage. The choice of the method may depend on the desired combination of the quantum dot system and the ligand or ligands.

With respect to aspect P37, a nanostructure and a Michael-donor compound are mixed, and a Michael-acceptor compound is added subsequently to the mixture. Initially the surface of the quantum dot is covered by a native ligand—e.g., oleylamine (OAm). By adding the Michael-donor compound first, this method maximizes the concentration of free amines, which may displace the OAm more efficiently. As a result, this method may increase the degree of the ligand exchange relative to similar methods (i.e., more completely replacing OAm by the new ligand).

With respect to aspect P38, a nanostructure and Michael-acceptor compound are mixed, and a Michael-donor compound is added subsequently to the mixture. If the native ligand (e.g., OAm) is bound very strongly to the quantum dot surface, then adding the Michael-acceptor compound first may be necessary in order to displace it. Excess Michael-acceptor compound would initially react sacrificially with OAm to form a non-binding adduct. Removal of the OAm would leave open sites on the quantum dot surface where the new ligand could bind.

With respect to aspect P39, a Michael-donor compound and a Michael-acceptor compound are mixed, and a nanostructure is added subsequently to the mixture. The advantage of this method is its simplicity and robustness. Here the Michael-donor and Michael-acceptor compounds react first to create a well-defined ligand species. This ligand species can then displace the native ligand in a direct ligand-exchange reaction. In this method there would be fewer possible side reactions and therefore fewer sources of variation.

Each Michael-donor compound herein is a compound having an amine group and a thiol group. A Michael-donor compound can be selected from a di-amine, a di-thiol or a mono-amine and mono-thiol. The thiol group is often more reactive towards Michael addition, and depending on the kind of quantum dot, the di-amine of di-thiol may be preferable, but more generally the groups can be selected depending on the kind of nanoparticle, as explained later. For example, an amine may bind more strongly to the surface of AIGS quantum dot, and the thiol may bind more strongly to surface of a ZnSe, ZnS, or CdS nanostructure, etc.

In some examples the most preferable structural unit of the Michael-donor compound is the di-amine or di-thiol structure. These units allow the Michael addition reaction to proceed and also leave a free amine or thiol group that can bind to the quantum dot surface. In these and other examples, the rest of chemical structure of the Michael-donor compound may vary.

The Michael-acceptor compound is selected to be compatible with the Michael-addition reaction and to provide good solubility in the target solvent. With respect to the first condition, what matters is the presence of the double bond and the X—C═O group next to it, which activates the double bond to act as an efficient Michael acceptor. This is why X may be O or NH. With respect to the second condition, R² and R³ may have a chemical character that ensures that the final ligand will provide good solubility in the target solvent system. The specific design rules will then depend on the properties of the solvent. For example, for more polar solvents (e.g., acrylate monomer formulations), R² or R³ include polar subunits—e.g., ether linkages, hydroxyl groups, etc., in order to provide relatively high solubility to the quantum dots.

In some examples, the Michael-donor compound is a compound of Formula (II):

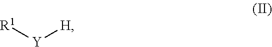

and the Michael-acceptor compound is a compound of Formula (III):

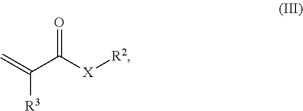

and the adduct is according to Formula (I):

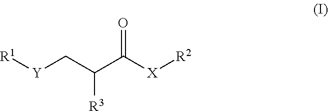

wherein:
Y is —NH— or —S—, and R¹ is selected from the group consisting of:
(i) Z—(C₁-C₆ alkylene)(C₃-C₈ cycloalkylene)(C₁-C₆ alkylene)-,
(ii) Z—(C₁-C₆ alkylene)(4- to 7-membered heterocyclylene)(C₁-C₆ alkylene)-,
(iii) Z—(C₁-C₆ alkylene)(C₆-C₁₄ arylene)(C₁-C₆ alkylene)-,
(iv) Z—(C₃-C₈ cycloalkylene)(C₁-C₆ alkylene)(C₃-C₈ cycloalkylene)-,
(v) polyalkyleneiminyl, and
(vi) Z—(C₁₋₁₂ alkylene)-; or
   Y is absent, R¹ is (vii) Z—(C₁-C₆ alkylene)(4- to 7-membered heterocyclylene)-, and a nitrogen atom in the heterocyclylene provides a point of attachment of R¹ to a carbon in (I) which is β to a carbonyl;
      wherein Z is an amino or —SH; and
      wherein:
R³ is selected from the group consisting of hydrogen and C₁-C₆ alkyl, and R² is selected from the group consisting of:
(i) C₁-C₁₂ alkyl, substituted with zero, one, two, or three groups independently selected from C₁-C₄ alkyl, C₁-C₆ alkoxy, phenoxy, amino, halo, nitro, cyano, and hydroxy;
(ii) C₃-C₈ cycloalkyl, substituted with zero, one, two, or three groups independently selected from C₁-C₄ alkyl, C₁-C₆ alkoxy, amino, halo, nitro, cyano, and hydroxy; and
(iii) polyalkylene glycol, wherein zero, one or more units of the polyalkylene glycol chain is substituted with one or two C₁-C₄ alkyl groups; or
   R² is (iv) C₁₋₁₂alkylene-methacrylate or C₁₋₁₂alkylene-methacrylamide, and R³ is hydrogen, the C₁₋₁₂alkylene is substituted with zero, one or more groups independently selected from C₁-C₄ alkyl, C₁-C₆ alkoxy, phenoxy, amino, halo, nitro, cyano, and hydroxy; and
      wherein X is —NH— or —O—.

The reader will note that R¹ and Z groups are indicated for compounds (I) and (II) above. The (ii)-(vii) of R¹ are similar Michael addition reaction as the (i) of R¹. The Z of Formula (I) or Formula (II) effect for the reactivity of the Michael donor. The (ii)-(iv) of R² are similar Michael addition reaction as the (i) of R².

### III. COMPOSITIONS, FILMS, DEVICES, USES

In some examples there is provided a composition comprising: (a) a particle comprising a luminescent nanostructure and a ligand, wherein the ligand comprises at least one compound according to Formula (I); and (b) a carrier.

In some examples, there is provided composition comprising: (a) a particle comprising a luminescent nanostructure and a ligand; and (b) a carrier comprising a curable acrylate monomer, wherein the ligand comprises one or more acrylate, methacrylate, acrylamide or methacrylamide groups which are cross-linkable with the acrylate monomer of the carrier on curing.

In further examples, there is provided a composition comprising: (a) a particle comprising a luminescent nanostructure and a ligand; and (b) a carrier comprising a cured acrylate polymer, wherein the ligand is cross-linked with the acrylate polymer of the carrier. In some such cases, the ligand comprised one or more acrylate, methacrylate, acrylamide or methacrylamide groups which cross-linked with the acrylate of the carrier on curing.

In further examples, there is provided a method of making a composition comprising: (a) mixing: (i) a particle comprising a luminescent nanostructure and a ligand comprising one or more of an acrylate, methacrylate, acrylamide or methacrylamide group, and (ii) a carrier comprising an acrylate monomer; and (b) curing at least some of the acrylate monomer of the carrier to form an acrylate polymer cross-linked with the ligand.

In some examples, the carrier is a liquid and comprises at least one additive. In some examples, suitable additives are photoinitiators. In some examples, the additive is triphenyl phosphite, pentaerythritol tetrakis[3-(3,5-di-tert-butyl-4-hydroxyphenyl)propionate, isopropylthioxanthone, 2,4-diethylthioxanthone, or 3-(trimethoxysilyl)propyl acrylate.

In some examples, the carrier is liquid.

In some examples, the carrier comprises a curable acrylate monomer. Non-limiting examples of acrylate monomers are selected from methyl (meth)acrylate, ethylene glycol phenyl (meth)acrylate, di(ethylene glycol) methyl ether (meth)acrylate, diethylene glycol monoethyl ether acrylate, ethylene glycol methyl ether (meth)acrylate, 1,3-butylene glycol di(meth)acrylate, polyethylene glycol di(meth)acrylate, 1,6-hexanediol diacrylate, isobornyl acrylate, 2-phenoxyethylacrylate, 4-hydroxybutylacrylate, 2-hydroxy-3-phenoxypropylacrylate, or combinations thereof. In some examples, the acrylate monomers are isobornyl acrylate, 2-phenoxyethylacrylate, 4-hydroxybutylacrylate, 2-hydroxy-3-phenoxypropylacrylate, or a combination thereof.

In some examples, the liquid carrier additionally comprises a solvent.

In some examples, the carrier is solid. In some examples, a solid carrier may be formed from a liquid carrier. The conversion of liquid carrier to solid carrier may involve curing monomers in the carrier to form a polymer, for example by UV or heat curing.

In some examples, the carrier comprises a cured acrylate polymer. Non-limiting examples of cured acrylates are poly(methyl (meth)acrylate), poly(ethylene glycol phenyl (meth)acrylate), poly(di(ethylene glycol) methyl ether (meth)acrylate), poly(diethylene glycol monoethyl ether acrylate), poly(ethylene glycol methyl ether (meth)acrylate), poly(1,3-butylene glycol di(meth)acrylate), poly(polyethylene glycol di(meth)acrylate), poly(1,6-hexanediol diacrylate), poly(isobornyl acrylate), poly(tetrahydrofurfuryl acrylate), poly(lauryl acrylate), poly(tricyclodecane dimethanol diacrylate), poly(glycerol triacrylate), poly(1,1,1-trimethylolpropane triacrylate), poly(pentaerythritol tetraacrylate), poly(bistrimethylolpropane tetraacrylate), poly(dipentaerythritol pentaacrylate), poly(pentaerythritol triacrylate), poly(pentaerythritol tetracrylate), poly(trimethylolpropane triacrylate), poly(dipentaerythritol pentaacrylate ester), poly(isobornyl methacrylate), poly(tetrahydrofurfuryl methacrylate), poly(lauryl methacrylate), poly(tricyclodecane dimethanol dimethacrylate), poly(glycerol trimethacrylate), poly(1,1,1-trimethylolpropane trimethacrylate), poly(pentaerythritol tetramethacrylate), poly(bistrimethylolpropane tetramethacrylate), poly(dipentaerythritol pentamethacrylate), poly(pentaerythritol trimethacrylate), poly(pentaerythritol tetramethcrylate), poly(trimethylolpropane trimethacrylate), poly(dipentaerythritol pentamethacrylate ester), poly(1,6-hexanediol dimethacrylate), poly(1,4-butanediol diacrylate), poly(1,9-nonanediol diacrylate), poly(1,4-butanediol dimethacrylate), poly(1,9-nonanediol dimethacrylate), poly(2-phenoxyethylacrylate), poly(4-hydroxybutylacrylate), poly(2-hydroxy-3-phenoxypropylacrylate), or combinations thereof. In some examples, the cured acrylates are poly(isobornyl acrylate), poly(2-phenoxyethylacrylate), poly(4-hydroxybutylacrylate), poly(2-hydroxy-3-phenoxypropylacrylate), or a combination thereof.

In some examples, the composition comprising a solid carrier comprises between about 70 wt % and about 90 wt % of one or more cured polymers. In some examples, the composition comprising a solid carrier comprises between about 70 wt % and about 85 wt %, about 70 wt % and about 80 wt %, about 75 wt % and about 90 wt %, or about 75 wt % and about 85 wt %, about 75 wt % and about 80 wt % of one or more cured polymers.

In some examples, the composition comprising a solid carrier comprises particles and cured polymers, wherein the weight ratio of particles to cured polymers is between about 1:9 and about 1:4. In some examples, the composition comprising a solid carrier comprises particles and cured polymers, wherein the weight ratio of particles to cured polymers is between about 1:4 and about 1:6.

In some examples, the composition comprising a solid carrier comprises between about 10 wt % and about 30 wt % particles. In some examples, the composition comprising a solid carrier comprises between about 10 wt % and about 20% particles.

In some examples, the composition provided as a film or layer. In some such examples, the film exhibits a photon conversion efficiency (PCE) of from about 20% to about 35%, or from about 25% to about 30%.

In some examples, the film has a thickness of 500 μm or less, 100 μm or less, or 50 μm or less. In some examples, the film has a thickness of about 15 μm or less.

In some examples, the film further comprises one or more barrier layers immediately adjacent to the film that have low oxygen and moisture permeability and protect the nanostructures from degradation.

In some examples, the film does not comprise a barrier layer. Without wishing to be bound by theory, it is believed that the ligands of examples herein are able to disperse nanostructures in polar solvents, resins, and/or matrices, yet still provide adequate protection from degradation in the presence of oxygen and/or water. It is believed that strong hydrogen bonding between the ligands increases the cohesive energy of the ligand corona, allowing it to act as a more efficient physical barrier towards oxygen and reactive oxygen species (ROS).

In some examples, the film has a photon conversion efficiency (PCE) of about 25%. In some examples, the film has a PCE of about 5%, about 10%, about 15%, about 20%, about 25%, about 30%, about 35%, about 40%, about 45%, or about 50%.

In some examples, the film has a PCE of from about 10% to about 40%, from about 15% to about 40%, from about 20% to about 40%, from about 25% to about 40%, from about 30% to about 40%, from about 35% to about 40%, from about 10% to about 35%, from about 15% to about 35%, from about 20% to about 35%, from about 25% to about 35%, from about 30% to about 35%, from about 10% to about 30%, from about 15% to about 30%, from about 20% to about 30%, from about 25% to about 30%, from about 10% to about 25%, from about 15% to about 25%, from about 20% to about 25%, from about 10% to about 20%, from about 15% to about 20%, or from about 10% to about 15%.

In some examples, the film retains about 95% of its PCE after exposure to air and 20 lux yellow light for 24 hours relative to its PCE before exposure to air or light. In some examples, the film retains about 80%, about 85%, about 90%, about 93%, about 95%, about 96%, about 97%, about 98%, or about 99% of its PCE after exposure to air and 20 lux yellow light for 24 hours relative to its PCE before exposure to air or light.

In some examples, the film retains from about 85% to about 99%, from about 90% to about 99%, from about 93 to about 99%, or from about 95% to about 97% of its PCE after exposure to air and 20 lux yellow light for 24 hours relative to its PCE before exposure to air or light.

A film in accordance with examples described herein is fabricated for example by depositing a composition with a liquid carrier described therein, then hardening and/or solidifying the composition from its liquid form to a more solid form. This hardening and/or solidifying is, e.g., done by UV curing and/or heating.

Depositing the composition in liquid form is for example done by printing, e.g., using an inkjet printing technique. Though in other examples depositing may involve extrusion or spreading the liquid form composition before solidifying and/or hardening.

Once the liquid form composition has solidified and/or hardened the composition may then be referred to as a film or layer. As will be referred to later, such a film or layer may be referred to as a so-called quantum dot enhancement film (QDEF) or in other examples as a quantum dot colour conversion (QDCC) film. Such a QDEF is, e.g., a film with an extent for covering all sub-pixels and/or pixels of a display device. Whereas such a QDCC, e.g., comprises an array or plurality of films each corresponding respectively with a light source of the display device; so one such film may correspond with one such light source and hence sub-pixel. In such a QDCC film, the composition, e.g., comprises: a first region comprising a first population of a plurality of particles, the first population having first luminescent nanostructures for emitting light of a first colour (e.g., red), and a second region comprising a second population of the plurality of particles, the second population having second luminescent nanostructures for emitting light of a second colour (e.g., green). Hence a first film of the array or plurality of films is, e.g., the first region with the first population and a second film of the array or plurality of films is, e.g., the second region with the second population. Ink jet printing is useful for depositing such films, as first an array of films of a first type (e.g., with nanostructures for emitting red light) can be printed, then an array of films of a second type (e.g., with different nanostructures than the first type of film, and for emitting green light) can be printed, then if required one or more array of films of further types (e.g., with different nanostructures than those of the first and second type of film). Such an array of films hence can be printed to have regions corresponding respectively with red, green and blue sub-pixels of the display device.

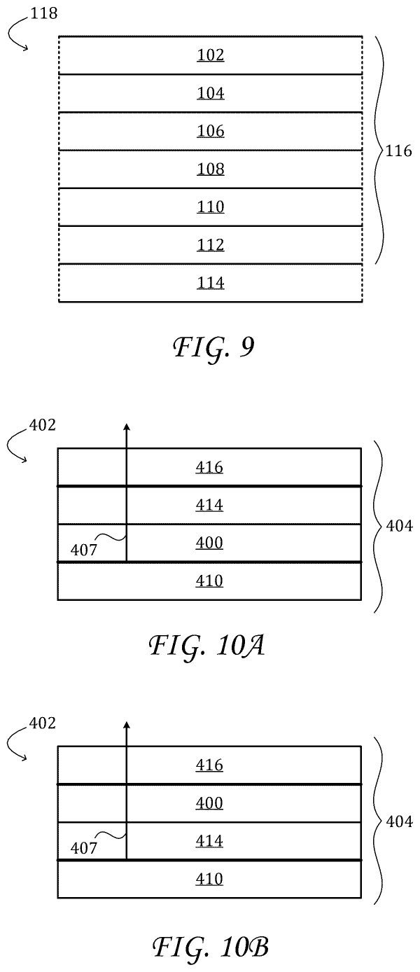

In further examples, such as those of FIG. 9, instead of a film such as a QDEF or QDCC film, the particles described herein with the ligands of examples are incorporated within a light source 118 itself. Such a light source may produce light by electroluminescence, with the nanostructures emitting light as a consequence of an electric current applied across the nanostructures. Such a light source 118 may for example be referred to as a light emitting diode, and for example comprises: (a) a first electrode 102; (b) a second electrode 112; and (c) a layer 108 between the first electrode and the second electrode. In some examples not shown the layer is in contact with one or each of the first electrode and the second electrode. The layer 108 comprises particles of one or more example described herein (comprising a nanostructure with ligands of one or more example herein). Such a layer may be referred to as an emission layer. And such a layer is for example a film or layer described earlier, and fabricated, e.g., by inkjet printing. An array of such light sources may be fabricated—e.g., by printing at least the layer of each light source in a similar way to forming the array of films described above in the context of a QDCC film.

The light source 118, in some examples, such as those of FIG. 9, comprises one or more additional layer (104, 106, 110, 112) between the first electrode and the second electrode, for example, a hole injection layer 104, a hole transport layer 106, an electron transport layer 110 and/or an electron injection layer 112 as the skilled person will understand. In some examples, at least one of the layers between the first electrode and the second electrode comprises an organic material.

As the skilled person will understand, when voltage is applied to the first electrode and the second electrode, holes injected at the first electrode move to the emission layer via the hole injection layer and/or the hole transport layer, and electrons injected from the second electrode move to the emission layer via the electron transport layer. The holes and electrons recombine in the emission layer to generate excitons.

In some examples, the light source 118 comprises a substrate 114. The substrate is, e.g., glass or a flexible material such as polyimide, or polyethylene terephthalate. In some examples, the substrate has a thickness of between about 0.1 mm and about 2 mm. In some examples, the substrate is a glass substrate, a plastic substrate, a metal substrate, or a silicon substrate.

In some examples, the first electrode is disposed on the substrate. In some examples, the first electrode is a stack of conductive layers. In some examples, the first electrode is deposited as a thin film using any known deposition technique, such as, for example, sputtering or electron-beam evaporation. In some examples, the first electrode is an anode or a cathode.

In some examples, the first electrode is an anode and the second electrode is a cathode, in other examples, the first electrode is a cathode and the second electrode is an anode. The first electrode and/or the electrode layer may comprise at least one of comprises indium tin oxide (ITO), indium zinc oxide (IZO), tin dioxide (SnO₂), zinc oxide (ZnO), magnesium (Mg), aluminum (Al), aluminum-lithium (Al—Li), calcium (Ca), magnesium-indium (Mg—In), magnesium-silver (Mg—Ag), or silver (Ag), gold (Au), or a mixtures thereof. In some examples, the second electrode is a stack of conductive layers. For example, the second electrode may include a layer of silver sandwiched between two layers of ITO (ITO/Ag/ITO).

In some examples, the light source 118 further comprises a hole injection layer 104. In some examples not shown, the hole injection layer 104 is deposited on the first electrode. In some examples, the hole injection layer is deposited by vacuum deposition, spin-coating, printing, casting, slot-die coating, or Langmuir-Blodgett (LB) deposition.

In some examples, the hole injection layer comprises copper phthalocyanine, 4,4′,4″-tris[(3-methylphenyl)phenylamino]triphenylamine (m-MTDATA), 4,4′,4″-tris(diphenylamino) triphenylamine (TDATA), 4,4′,4″-tris[2-naphthyl(phenyl)amino]triphenylamine (2T-NATA), polyaniline/dodecylbenzenesulfonic acid, poly(3,4-ethylenedioxythiophene)/polystyrene sulfonate) (PEDOT/PSS), polyaniline/camphor sulfonic acid, or polyaniline/poly(4-styrenesulfonate).

In some examples, the light source 118 further comprises transport layers (110 and 106) for the facilitation of the transport of electrons and holes affected by a generated electric field between the first electrode 102 and the second electrode 112. In some examples, the light source 118 comprises a first transport layer associated with the first electrode. In some examples, the first transport layer is a hole transport layer (and an electron and/or exciton blocking layer). In some examples, the first transport layer is deposited on the first conductive layer. In some examples, the first transport layer is deposited on the hole injection layer. In some examples, the first transport layer is substantially transparent to visible light.

In some examples, the first transport layer comprises a material selected from the group consisting of an amine, a triarylamine, a thiophene, a carbazole, a phthalocyanine, a porphyrin, or a mixture thereof. In some examples, the first transport layer comprises N,N′-di(naphthalen-1-yl)-N,N′-bis(4-vinylphenyl)-4,4′-diamine, poly[(9,9-dioctylfluorenyl-2,7-diyl)-co-(4,4′-(N-(4-sec-butylphenyl))diphenylamine)], and poly(9-vinylcarbazole).

In some examples, the light source further comprises a second transport layer. In some examples, the second transport layer is an electron transport layer (and a hole and/or exciton blocking layer). In some examples, the second transport layer contacts the emission layer. In some examples, the second transport layer is between the emission layer and the second conductive layer. In some examples, the second transport layer is transparent to visible light.

In some examples, the electron transport layer comprises at least one of zinc oxide or zinc magnesium oxide.

In examples, an apparatus comprises a composition as described previously, e.g., a film or liquid composition. Such apparatus may comprise a light source configured to emit light of one or more wavelength absorbed by the nanostructures. In some examples there is also a filter array comprising red light filters for transmitting red light, green light filters for transmitting green light, and blue light filters for transmitting blue light; and a light valve array. In other such examples the light source is a light emitting diode comprising the composition or film, e.g., as a layer (e.g., uppermost layer) of the light emitting diode located to receive light generated by the light emitting diode and output light from the nanostructures onwards. In this way each light source may be configured with the composition or film. Such a light source is for example an electroluminescent light source described above. Another apparatus with each light source having a corresponding film of examples described herein is for example an array of films each positioned to receive light emitted from a respective light source of a plurality of the light sources. Such a plurality or array is, e.g., provided as a layer separate from the light sources, and may be ink jet printed, and may be referred to as a QDCC film referred to earlier.

Further details of such apparatus will now be elaborated in more detail below.

Apparatus of examples herein is now described with reference to FIGS. 10A and 10B. The apparatus illustrated schematically in FIGS. 10A and 10B is a display device 402 which has functional elements which are configured to operate together to generate and output an image. Some such functional elements are stacked and hence are referred to collectively herein as a display stack 404. The display stack 404, e.g., has a light source 410 configured to emit light 407 (e.g., a light emitting diode (LED) or organic LED (OLED) backlight), a light valve array 414 (e.g., a liquid crystal display (LCD) panel) for modulating an amount of light received from the light source, and a filter array 416 (e.g., a colour filter array such as a red, green and blue sub-pixel filter array) for determining a colour of light output by the display device 402 (e.g., by each sub-pixel region of the device). A film 400 according to examples may be arranged between the light source 410 and the light valve array 414 (as illustrated in FIG. 10A), or may be located between the light valve array 414 and the filter array 416 (as illustrated in FIG. 10B, for example).

The display device 402 has a light source 410 positioned, e.g., to provide a back lit or edge-lit display device. The light source is, e.g., at least one of: an LED, an LED array, an organic LED (OLED), an OLED array, a laser, a laser array, or a lamp. The light source may be configured for illuminating multiple picture elements of the display device or there may be a multitude of light sources with each illuminating a single picture element. A picture element is, e.g., a sub-pixel or pixel of a display device. A display device typically comprises a plurality of picture elements independently controllable for the display device to display an image. The picture elements are arranged according to a pattern, e.g., as an array, matrix or grid, as the skilled person understands. A display device capable of displaying a colour image typically has a plurality of pixels, with each pixel comprising a plurality of sub-pixels; for example, a pixel comprises a red (R) sub-pixel, a green (G) sub-pixel and a blue (B) sub-pixel which together function as an RGB pixel. There may be additional, independently controllable, sub-pixels, e.g., a white (W) sub-pixel, to give an RGBW pixel.

In addition to such a light source, a display device comprises a light modulator configured to modulate light emitted by the light source, for displaying an image. The light modulator has an array of light modulator regions to modulate light. Each light modulator region of the array of light modulator regions corresponds with a respective picture element of the display device. So, for example, when viewing a viewing side of the display device for displaying an image to a user's eye, a perimeter of one light modulator region determines an extent of one picture element. The light source or light guide has an extent covered by the array of light modulator regions, so that each light modulator region can be illuminated by the light source. The light valve array 414 described previously is an example of such a light modulator.

A display device control system (not illustrated) is configured to control the array of light modulator regions for the display device to output an image. Each light modulator region is independently controllable, to modulate the amount of light transmitted through the modulator region to the viewing side, for each picture element. Thus one light modulator region may be switched to transmit less light (a darker state) than another light modulator region (a lighter state), so that with appropriate light modulation across the array of light modulator regions (and therefore the picture elements) the display device can display a desired image.

As the skilled person understands, a type of light modulator available uses liquid crystal (LC) molecules for light modulation. By applying an electric field of appropriate magnitude to electrodes of a light modulator region, an orientation of the LC molecules can be changed to modulate light output by a respective picture element, for displaying an image on a viewing side. A LC type light modulator has a polarizer layer to linearly polarize light input to the light modulator. The polarizing layer is on a substrate (e.g., glass). There is a circuitry layer on the substrate, connected to an array of electrodes (e.g., of indium tin oxide (ITO)) each of which is electrically insulated from each other and has an extent which determines a shape and size of each picture element. On the electrodes there is a layer comprising LC molecules, the LC molecules and their density in the layer selected to provide a required rotation of linearly polarized light in accordance with a magnitude of applied electric field. An alignment layer is in contact with the layer comprising LC molecules, to align the LC molecules in contact with the alignment layer with a particular orientation. There is another linear polarizer layer, for polarizing light exiting the light modulator, orientated to linearly polarize light at an orientation—e.g., perpendicular to the polarizer layer described above. On the another linear polarizer layer is a substrate (e.g., of glass). There is an electrode with an extent to cover more than one (e.g., all) picture elements, which may be referred to as a common electrode.

Each picture element also comprises a colour filter, in these examples between the alignment layer and the another linear polarizer layer. By appropriate choice of the colour filter for each picture element, an RGB pixel (of three sub-pixel picture elements) can be made with red filters to transmit red light, e.g., of a 630 nanometre wavelength, green filters to transmit green light, e.g., of a 532 nanometre wavelength, and blue filters to transmit blue light, e.g., of a 467 nanometre wavelength. An array of such colour filters is an example of the filter array described previously.

The display device has a display stack of functional elements including for example the light source, the QDEF and the filter array. In these examples there is also the light modulator, and starting from the lowest layer of the stack, there is a substrate (e.g., of glass), a light source circuitry layer on the substrate, and a plurality of LEDs as the light source connected to the circuitry layer. In a back-lit example, the plurality of LEDs is configured and positioned, e.g., as an array of LEDs overlapped by the light modulator, to illuminate the light modulator.

The density and positioning of the LEDs depends at least partly on the shape and size of the picture elements of the display, but also the illumination characteristics of each LED and any layers such as a diffuser or reflector for transmitting light from the LEDs to the light modulator. In some such examples, each LED of the plurality of LEDs of the illumination device is configured to illuminate a plurality of picture elements (e.g., 50 to 100 or several thousands) of the display device. Each plurality of picture elements can be considered a zone, with a so-called mini-LED configuration, with each zone in some examples being independently controllable compared with other zones. Switching different zones differently can improve contrast, e.g., by switching off one zone to give a darker black, and may be referred to as “local dimming”.

In other examples, which are edge-lit, instead of the array of LEDs there is a light guide overlapped by the light modulator and at least one LED of the plurality of LEDs is positioned along at least part of a perimeter of the light guide, to illuminate the light modulator via the light guide. In some such examples, or other examples, there is a light diffuser overlapped by the light modulator. Between the LEDs and the light modulator may be one or more layer, e.g., a diffuser to distribute light from the LEDs more evenly across the light modulator, and/or alignment layer (e.g, a so-called brightness enhancement film (BEF) using, e.g., prisms to align light from the LEDs with each picture element. Further such diffuser and/or alignment layers may be used. Various other functional elements may be used as the skilled person will appreciate, for example to modify light, e.g.: a so-called dual BEF (DBEF) (e.g., to polarise light for a liquid crystal light valve array and to reflect light not of the desired polarisation for the liquid crystal light valve array to the backlight for re-reflection towards the DBEF), a TFE layer described previously, a prismatic layer, a reflector, a partial reflector, a polariser, a diffuser, a barrier layer, an anti-reflector, a collimator.

The circuitry layers for the light source and light modulator are each connected to the display device control system, and are configured for control of the light source and the light modulator by the display device control system to output a desired image. The circuitry layer of the light modulator is for example configured for so-called active matrix control of the light modulator regions, by using a switching element (e.g., a thin film transistor (TFT)) per picture element, and appropriate application of electrical signals to the source and gate terminals of each TFT, to set each light modulator region to transmit a desired amount of light. The circuitry layer of the light source is configured to control light output by the LEDs, e.g., to switch on a particular zone of LEDs whilst leaving switched off other zones of LEDs. Depending on the number of LEDs and their layout, the circuitry layer of the light source may even comprise switching elements (e.g., TFTs) for active matrix control of the LEDs.

The display device control system is connected to the circuitry layers and the common electrode by signal lines. The display device control system has for example a data input for receiving data representative of one or more images for the display device to display. As the skilled person will appreciate, the display device control system comprises circuitry for (and based on data representative of an image to be displayed) determining and applying appropriate electrical signals to the electrodes of the light modulator and the LEDs of the light source.

As the skilled person will appreciate, the magnitude of voltage applied between the common electrode and the electrode of a light modulator region of a given picture element, and therefore the size of the applied electric field, determines a rotational orientation of the LC molecules through the picture element relative to the alignment set by the alignment layer and also relative to the linear polarizer layers. Thus the extent of light modulation of each light modulator region can be controlled, and in turn the amount of light transmitted which is either aligned with the alignment layer or at least partly rotated in orientation relative to the alignment layer.

As the skilled person will appreciate, further types of examples are envisaged which have a light modulator in combination with a light source, but which use a different technology (e.g., microelectromechanical (MEMs) or electrophoretic technology) than LC molecules for light modulation.

Examples are further envisaged where LEDs of the light source each correspond with a picture element respectively, and are controllable to modulate light output by each picture element, rather than using a separate light modulator in combination with the illumination device. For example, each sub-pixel may comprise a blue LED, and/or a plurality of sub-pixels may be illuminated by one white LED, or instead a green and a red LED. By appropriate control of each blue LED and the green and red LEDs, a colour of an image output by the display device can be adjusted.

The display device of examples described herein is, e.g., a display panel, display unit or display screen for apparatus such as: a television, a computer monitor, a tablet computing device, a laptop computing device, a mobile telecommunications device such as a smart phone, a portable (e.g., mobile) device, an electronic reader device, a watch, a satellite navigation device, a heads-up display device, a games console, a flexible display, an extended reality (XR) device, a virtual reality (VR) device, and/or an augmented reality (AR) device.

Hence the display device is for example incorporated into apparatus comprising: the display device, at least one processor; and at least one memory comprising computer program instructions, the at least one memory and the computer program instructions operable to, with the at least one processor, control the display device control system for controlling the display device to output an image.

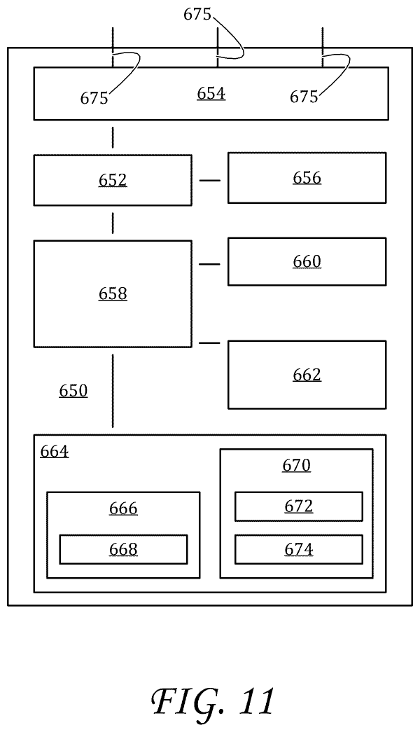

A system diagram illustrating an example of a basic hardware architecture of the system 650 is shown in FIG. 11, such as a laptop computing device. Note that in other implementations some of the components shown in FIG. 11 are not present; for example for a computer monitor implementation, the system storage and/or battery may not be present. The system 650 comprises: the display device 654; at least one processor 658 connected to and therefore in data communication with for example: a display device control system 652 (e.g., according to examples described earlier), a communications system 656, a user input system 660, a power system 662 and system storage 664. The display device control system is connected to and is therefore in data communication with the display device 654.

The display device control system 652 for example includes driver components, for use in applying a voltage to any of the picture elements, to address different such picture elements. In examples the light modulator regions of the picture elements are driven using an active-matrix control scheme and the display device control system is configured to control switching elements such as thin film transistors (TFTs) of the display device 654 via circuitry to control the picture elements. The circuitry may include signal and control lines. For example, the display device control system 652 may include display drivers such as display column drivers and display row drivers.

The at least one processor 658 herein is, for example: a general-purpose processor; a microprocessor; a digital signal processor (DSP); an application specific integrated circuit (ASIC); a field programmable gate array (FPGA); a programmable logic device; a discrete gate or transistor logic; discrete hardware components; or any suitable combination thereof configurable for the functions described herein. A processor may be a combination of computing devices such as: a DSP and a microprocessor; a plurality of microprocessors; a microprocessor in conjunction with a DSP core; or any other such configuration. The processor 658 may be coupled, via one or more buses, to read information from or write information to a memory of the storage. The processor 658 may additionally, or in the alternative, contain a memory, such as a processor register.

The communications system 656 is for example configured for the system 650 to communicate with, for example: a computing device via a data network; a computer network such as the Internet; a local area network (LAN); a wide area network (WAN); a telecommunications network, a wired network, a wireless network, or an other network. The communications system may comprise: an input/output (I/O) interface such as a universal serial bus (USB) connection, a Bluetooth connection, or infrared connection; or a data network interface for connecting the apparatus to a data network such as any of those described above. Content data as described later may be transferred to the system via the communications system.

The user input system 660 may comprise an input device for receiving input from a user of the system. Example input devices include, but are not limited to, a keyboard, a rollerball, buttons, keys, switches, a pointing device, a mouse, a joystick, a remote control, an infrared detector, a voice recognition system, a bar code reader, a scanner, a video camera (possibly coupled with video processing software to, e.g., detect hand gestures or facial gestures), a motion detector, a microphone (possibly coupled to audio processing software to, e.g., detect voice commands), a VR glove, AR glove, a haptic input device, a computer vision device, a simultaneous localization and mapping (SLAM) device, an eye-tracking device, a hand-tracking device, or other device capable of transmitting information from a user to the device. The input device may also take the form of a touch-screen associated with the display device 654, in which case a user responds to prompts on the display device 654 by touch. The user may enter textual information through the input device such as the keyboard or the touch-screen.

The system may also include a user output system (not illustrated) including for example an output device for providing output to a user of the system. Examples include, but are not limited to, a printing device, an audio output device including for example one or more speakers, headphones, earphones, alarms, or haptic output devices. The output device may be a connector port for connecting to one of the other output devices described, such as earphones.

The power system 662 for example includes power circuitry for use in transferring and controlling power consumed by the system. The power may be provided by a mains electricity supply or from a battery (not shown), via the power circuitry. The power circuitry may further be used for charging the battery from a mains electricity supply.

The storage 664 includes a memory, for example at least one volatile memory 666 and non-volatile memory 670 and may comprise a non-transitory computer readable storage medium. The volatile memory may for example be a Random Access Memory (RAM). The non-volatile (NV) memory may for example be a solid-state drive (SSD) such as Flash memory or Read Only Memory (ROM). Further storage technologies may be used, for example magnetic, optical or tape media, compact disc (CD), digital versatile disc (DVD), Blu-ray or other data storage media. The volatile and/or non-volatile memory may be removable or non-removable. Any of the memories may store data for controlling the system. Such data may for example be in the form of computer readable and/or executable instructions, for example computer program instructions. Therefore, the at least one memory and the computer program instructions may be operable to, with the at least one processor, control the display device control system for controlling the display device 654 to output an image.

In the example of FIG. 11, the volatile memory 666 stores for example display device data 668 which is indicative of an image to be provided by the system. The processor 658 may transmit data, based on the display device data 668, to the control system 652 which in turn outputs signals to the display device for applying voltages to the picture elements, for displaying an image 675. The non-volatile memory 670 stores for example program data 672 and/or content data 674. The program data is for example data representing computer executable instructions, for example in the form of computer software, for the system to run applications or program modules for the system or components or systems of the system to perform certain functions or tasks, and/or for controlling components or systems of the system. For example, application or program module data includes any of routines, programs, objects, components, data structures or similar. The content data is for example data representing content for example for a user; such content may represent any form of media, for example text, at least one image or a part thereof, at least one video or a part thereof, at least one sound or music or a part thereof. Data representing an image, or a part thereof is for example representative of an image to be provided by at least one picture element of the display device. Such data may include content data of one type but may instead include a mixture of content data of different types, for example a movie may be represented by data including at least image data and sound data.

### IV. EXAMPLES RELATING TO COMPOUNDS

### Example 1: Synthesis of Michael Adduct Ligands from Acrylate and Diamine Precursors

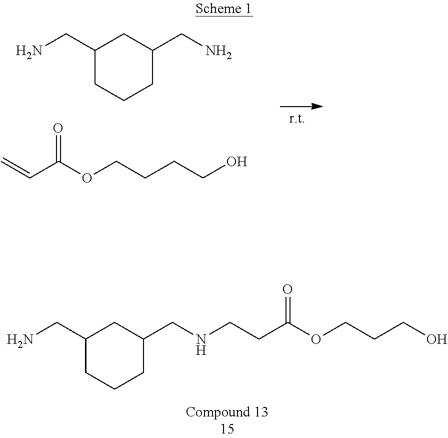

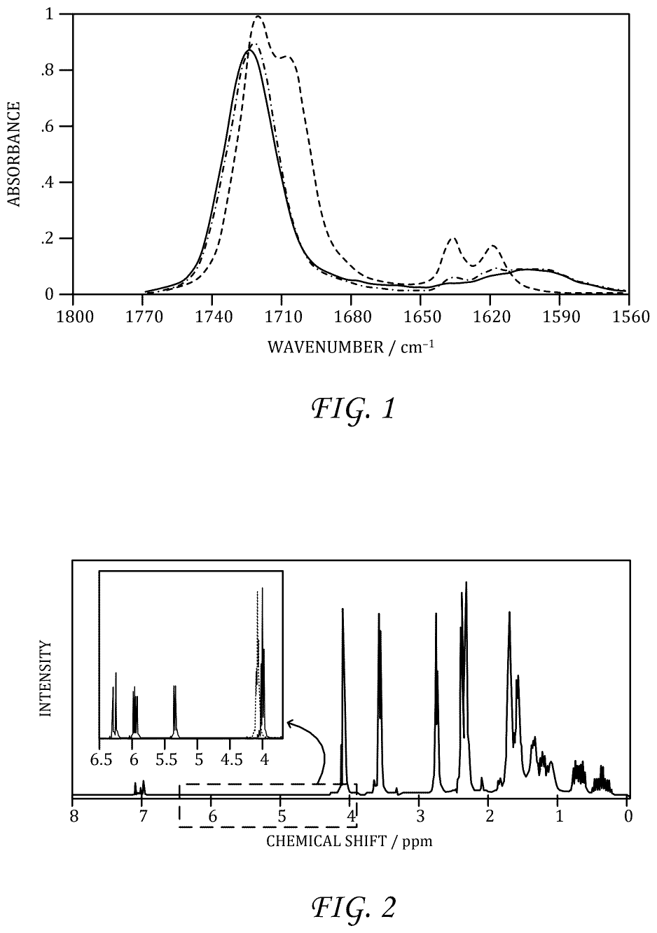

4-Hydroxybutylacrylate (HBA) and 1,3-bis(aminomethyl)cyclohexane (CHBMA) were mixed in a 1:1 molar ratio at room temperature to form a Michael adduct, Compound 15, as shown in Scheme 1. The conversion of the starting materials to product was observed using Fourier transform infrared spectroscopy (FT-IR) and proton nuclear magnetic resonance spectroscopy (¹H NMR), as shown in FIGS. 1 and 2, respectively. The dashed line in FIG. 1 corresponds to HBA; the dot-dashed line corresponds to the mixture of HBA+CHBMA after 1 minute; and the solid line corresponds to the mixture after 5 minutes. The disappearance of the sharp peaks in the 1610 to 1650 cm-1 range indicate complete conversion of the initial acrylate species. In FIG. 2, the solid line of the inset corresponds to the HBA/CHBMA adduct; the broken line corresponds to HBA.

Compounds 22 and 29 were synthesized in the same manner, as shown in Scheme 2.

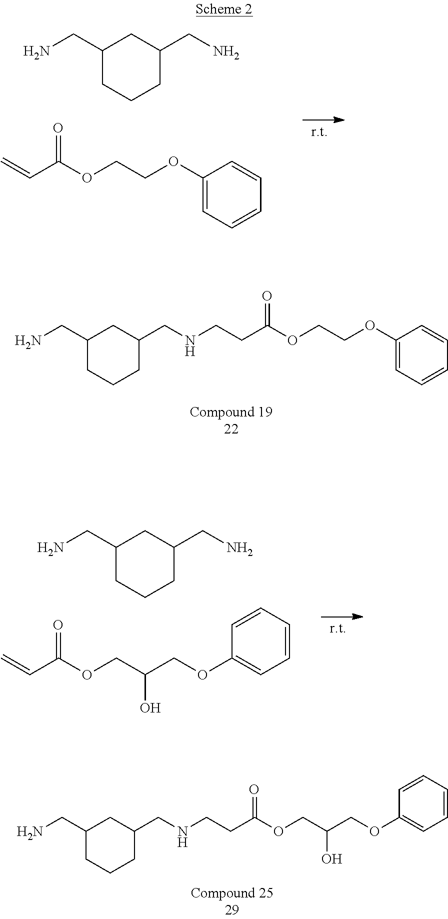 [SMILES: NCC1CCCC(CN)C1 ; C=CC(=O)OCCOc1ccccc1 ; NCC1CCCC(CNCCC(=O)OCCOc2ccccc2)C1 ; NCC1CCCC(CN)C1 ; C=CC(=O)OCC(O)COc1ccccc1 ; NCC1CCCC(CNCCC(=O)OCC(O)COc2ccccc2)C1]

Another example reaction is the reaction between N-butylacrylamide and (1,3-bis(aminomethyl)cyclohexane) (CHBMA) to produce as ligand S1 shown below. Ligand exchange was carried out successfully using this ligand according to the method of aspect P38 (analogous to the reaction of Scheme 1 above). The reaction produced a QD solution that was colloidally stable.

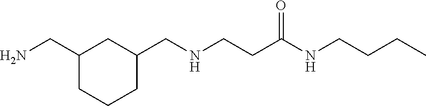 [SMILES: CCCCNC(=O)CCNCC1CCCC(CN)C1]

S1: The ligand formed by the reaction of CHBMA with N-butylacrylamide.

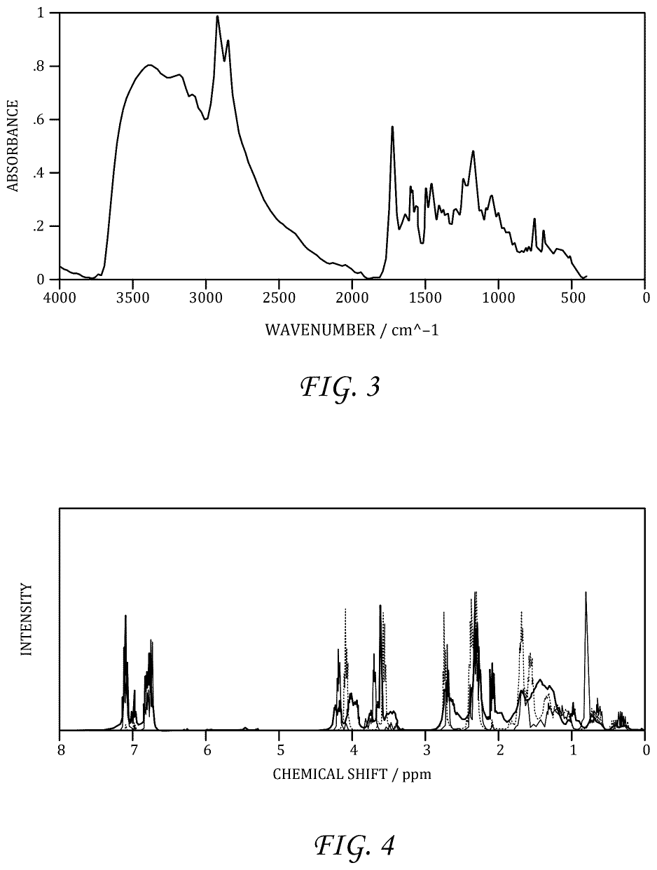

Example 2: Ligand Exchange to Quantum Dots. Ligand exchange was carried out on AgInGaS/GaS quantum dots to replace the native ligands with a combination of Compound 15 and Compound 22. Ligand exchange was observed by FT-IR and ¹H NMR, as shown in FIGS. 3 and 4, respectively. With respect to FIG. 4, the presence of bound Michael addition adducts is indicated by the presence of broader resonances coinciding with those found in the spectra of the ex-situ free adducts. The residual presence of the native oleylamine ligand is also observed. The fine broken line in FIG. 4 corresponds to the HBA/CHBMA adduct; the fine solid line corresponds to the PhEA/CHBMA adduct; the heavier solid line corresponds to QDs bound to a combination of the two adducts.

Ligand exchange could be carried out either ex situ, i.e, after the Michael adduct ligand had been synthesized, or in situ, i.e., in the presence of the starting materials for the Michael adduct ligand.

Example 3: Films Comprising Quantum Dots with Michael Adduct Ligands. AgInGaS/GaS quantum dots were ligand-exchanged using either the in situ or ex situ method described in Example 2 and incorporated into a carrier comprising 1,6-hexanediol diacrylate and a photoinitiator, and the carrier was then formed into a film 9.5 μm thick. The film was cured using UV light. The photoconversion efficiency (PCE) of the resulting films was measured both initially and after 24 h exposure to air and 20 lux yellow light. The results are shown in Table 2. Films comprising Michael adduct ligands demonstrated up to 96.7% retention of PCE after 24 h exposure to air and 20 lux yellow light.

TABLE 2

| | Post | | | |
|---|---|---|---|---|
| | 180° C. | Post Light | PCE | |
| | Bake | Exposure | Retention | |
| | Green | Green PCE | in Light | Additional |
| Ligand(s) | PCE (%) | (%) | (%) | Additive |
| Jeffamine, mixture | 27.4 | 22.5 | 82.1 | — |
| of CHBMA and | 28.5 | 22.4 | 78.5 | TPP |
| IBOA to form | 28.9 | 22.8 | 79.1 | DETX |
| Compound 1 in | | | | |
| situ | | | | |
| mixture of CHBMA, | 28.2 | 26.9 | 95.5 | — |
| PhEA, and HBA to | 28.8 | 27.9 | 97.1 | ITX |
| form Compounds 15 | 27.6 | 26.7 | 96.7 | DETX |
| and 22 in situ | | | | |
| mixture of CHBMA, | 30.1 | 26.3 | 87.5 | — |
| PhEA, and HBA to | 30.0 | 28.2 | 93.9 | ITX |
| form Compounds 15 | 29.5 | 28.4 | 96.5 | ITX + |
| and 22 in situ | | | | TMPSA |
| Compounds 15 and | 29.5 | 27.5 | 93.2 | — |
| vf ormed ex situ | 26.4 | 24.3 | 88.9 | — |
| from CHBMA, | 28.0 | 26.2 | 93.6 | — |
| PhEA, and HBA | | | | |
| mixture of CHBMA | 28.2 | 22.5 | 82.1 | — |
| and HOPhEA to | 28.8 | 22.4 | 78.5 | TPP |
| form Compound 29 | 27.6 | 22.8 | 79.1 | DETX |
| in situ | | | | |
| Compound 29 | 30.1 | 26.9 | 95.5 | — |
| formed ex situ | 30.0 | 27.9 | 97.1 | ITX |
| from CHBMA | | | | |
| and HOPhEA | 29.5 | 26.7 | 96.7 | DETX |

Abbreviations:
IBOA = isobornyl acrylate;
HOPhEA = 2-hydroxy-3-phenoxypropyl acrylate;
TPP = triphenyl phosphite;
ITX = isopropylthioxanthone;
DETX = 2,4-diethylthioxanthone;
TMPSA = 3-(trimethoxysilyl)propyl acrylate.

In particular, films comprising quantum dots without the above-referenced Jeffamine, a polyetheramine commonly used as a ligand, retained a significantly higher percentage of initial PCE compared to films comprising quantum dots with Jeffamine. Without wishing to be bound by theory, it is believed that using Jeffamine ligands, which have ether linkages having an abstractable hydrogen group, result in radical formation close to the QD surface and subsequent radical-mediated damage to the QD surface. The presently disclosed Michael adduct ligands are less likely to encounter this issue, as the ligands are expected to bind through the amine portion of the ligand, thereby creating a buffer between the QD surface and the oxygen-containing portion of the ligand. Further, it is believed that Michael adduct ligands which are capable of hydrogen bonding, e.g., when the ligand includes a hydroxyl group, are added together to a population of QDs, strong hydrogen bonding increases the cohesive energy of the ligand corona, allowing it to act as a more efficient physical barrier towards oxygen and reactive oxygen species (ROS). FIG. 5 shows that when a hydroxyl is present in the ligand shell of quantum dots, e.g., when Compound 15 is a ligand, films comprising said quantum dots may have a higher PCE after 24 h exposure to air and 20 lux yellow light.

Example 4: Acrylate films including quantum dots having ligands with methacrylate group. 1,3-Bis(aminomethyl)cyclohexane (CHBMA) and 3-(acryloyloxy)-2-hydroxypropyl methacrylate (AHPMA) were mixed in a 1:1 molar ratio at room temperature to form a Michael adduct, Compound 113, as shown in Scheme 3 as “CAH”.

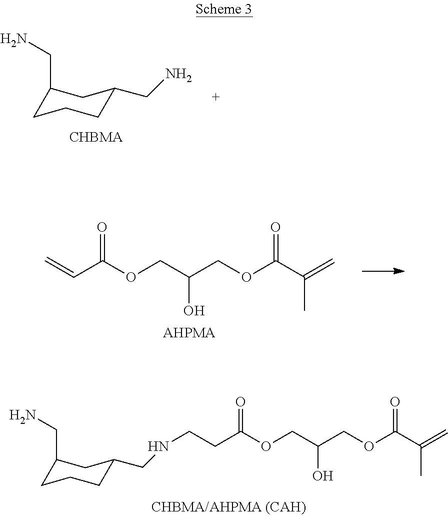

The resulting adduct was mixed with 1,6-hexandiol diacrylate (HDDA) and the mixture was cured under UV light. Cross-linking bonds form between the methacrylate group of CAH and the acrylate groups of the HDDA.

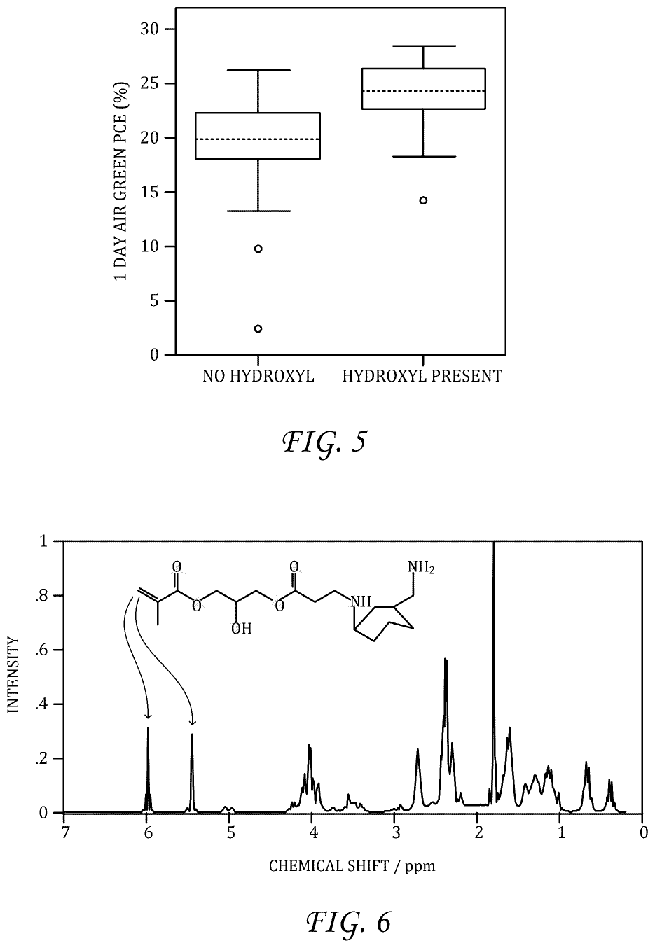

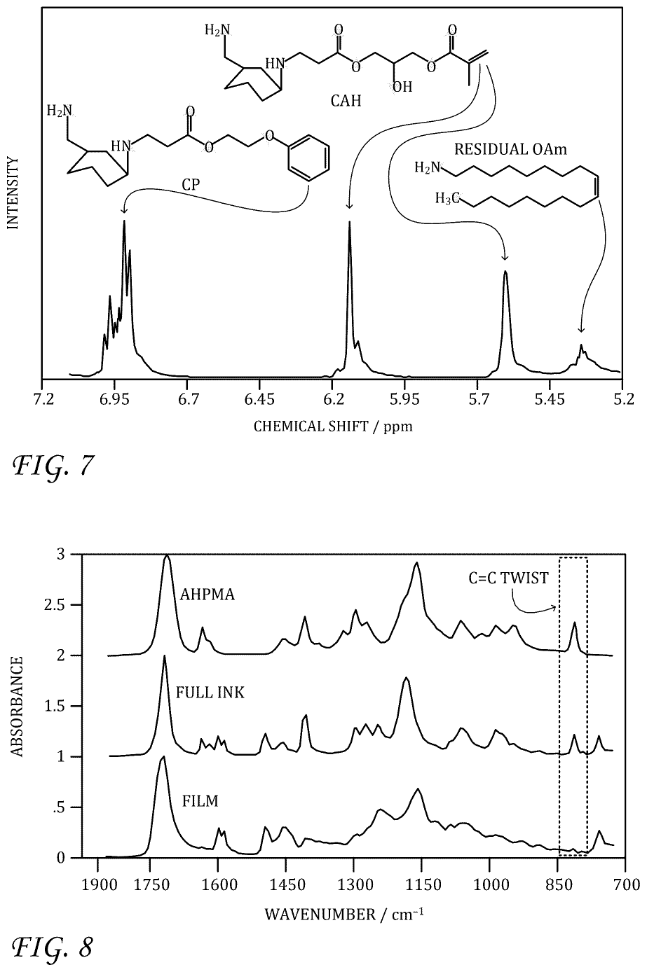

The structural evolution described above can be demonstrated using a combination of NMR and FTIR spectroscopy. First, ex-situ NMR studies of the reaction between CHBMA and AHPMA indicate the continued presence of the methacrylate group upon formation of the ligand. FIG. 6 shows the disappearance of the resonance associated with the acrylate group (indicating complete reaction with the amine), but the persistence of the peaks associated with the lower reactivity methacrylate group. Next, upon a ligand exchange that includes this system as part of the mix, the same peaks (now in a broadened form) indicate the presence of the CAH in the ligand shell. In FIG. 7 peaks corresponding to the methcrylate group are observed. Finally, upon film curing, FTIR indicates the almost quantitative consumption of peaks associated with the C═C bond in AHPMA, indicating the formation of covalent bods with the monomers in the ink that generate the polymeric matrix. In FIG. 8 the feature associated with the presence of methacrylate is almost completely absent in the spectrum of the film, indicating that this group has reacted with the monomer ink.

AHPMA includes both an acrylate and a methacrylate group. The above synthesis concept is analogous to “dual curing” approaches used in polymer engineering and relies on the fact that the acrylate group is more reactive than the methacrylate group. As a result, it is possible to employ this species to carry out a programmed two-step reaction scheme. The first reaction is Michael addition between the acrylate unit and an amine group on a diamine. The species that results has 1) an amine anchoring group allowing it to function as a ligand and 2) a terminal methacrylate group that can form covalent bonds with acrylate monomers such as HDDA.

Without wishing to be bound by theory, it is thought that the covalent bonds that form between the ligand and the surrounding polymer network can be expected to improve the robustness of the ligand shell as a physical barrier in two ways. First, by tethering the ligands to the polymer backbone of the film, the ligands are locked in place, preventing their dissociation and migration away from the QD surface. In addition, the new bonds that form increase the effective “crosslinking density” in proximity to the QD surface, which can reduce access to the QD surface for oxygen, water and other harmful species.

As the skilled artisan will understand, it is possible to cross-link the ligand to the surrounding polymer network without the ligand being the product of a Michael addition. This can be achieved through using a ligand with an acrylate, methacrylate, acrylamide or methacrylamide group, in conjunction with an acrylate monomer precursor to the polymer network.

In conclusion, aspect P1 of this disclosure is directed to a compound having Formula (I):

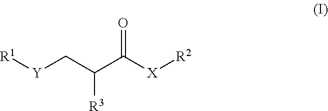

wherein the symbols R¹, R², R³, X, and Y are as defined hereinabove. Aspect P2 is directed to the compound of aspect P1 wherein Z is amino and Y is —NH—. Aspect P3 is directed to the compound of aspect P1 or P2, wherein R¹ is (amino)(C₁-C₆ alkylene)(C₃-C₈ cycloalkylene)(C₁-C₆ alkylene)-. Aspect P4 is directed to the compound of aspect P3, wherein R¹ is:

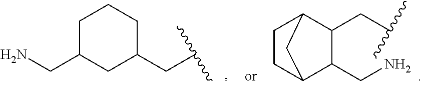 [SMILES: *CC1CCCC(CN)C1 ; *CC1C2CCC(C2)C1CN]

Aspect P5 is directed to the compound of aspects P1 or P2, wherein R¹ is (amino)(C₁-C₆ alkylene)(4- to 7-membered heterocyclylene)(C₁-C₆ alkylene)-. Aspect P6 is the compound of aspect P5, wherein R¹ is:

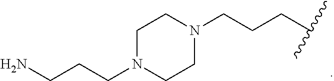 [SMILES: *CCCN1CCN(CCCN)CC1]

Aspect P7 is directed to the compound of aspects P1 or P2, wherein R¹ is (amino)(C₁-C₆ alkylene)(C₆-C₁₄ arylene)(C₁-C₆ alkylene)-. Aspect P8 is the compound of aspect P7, wherein R¹ is:

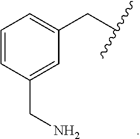 [SMILES: *Cc1cccc(CN)c1]

Aspect P9 is directed to the compound of aspects P1 or P2, wherein R¹ is (amino)(C₃-C₈ cycloalkylene)(C₁-C₆ alkylene)(C₃-C₈ cycloalkylene)-. Aspect P10 is the compound of aspect P9, wherein R¹ is:

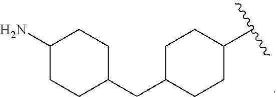 [SMILES: *C1CCC(CC2CCC(N)CC2)CC1]

Aspect P11 is directed to the compound of aspect P1, wherein R¹ is (amino)(C₁-C₆ alkylene)(4- to 7-membered heterocyclylene)- and Y is absent, and wherein a nitrogen atom in the heterocyclylene provides the point of attachment of R¹ to the carbon in Formula (I) that is β to the carbonyl group. Aspect P12 is the compound of aspect P11, wherein R¹ is:

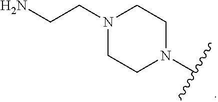 [SMILES: *N1CCN(CCN)CC1]

Aspect P13 is directed to the compound of aspects P1 or P2, wherein R¹ is polyalkyleneiminyl. Aspect P14 is the compound of aspect P13, wherein R¹ is polyethyleneiminyl. Aspect P15 is the compound of aspects P1 or P2, wherein R¹ is Z—(C₁₋₁₂ alkylene)-. Aspect P16 is the compound of aspect P15, wherein R¹ is:

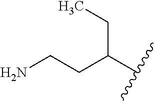 [SMILES: *C(CC)CCN]

Aspect P17 is directed to the compound of any one of aspects P1-P16, wherein R² is C₁-C₁₂ alkyl, substituted with zero one, two, or three groups selected from C₁-C₄ alkyl, C₁-C₆ alkoxy, phenoxy, amino, halo, nitro, cyano, and hydroxy. Aspect P18 is the compound of aspect P17, wherein R² is selected from the group consisting of:

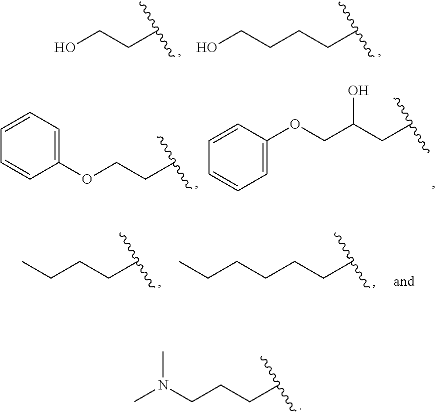

Aspect P19 is directed to the compound of any one of aspects P1-P16, wherein R² is C₃-C₈ cycloalkyl, substituted with zero one, two, or three groups selected from C₁-C₄ alkyl, C₁-C₆ alkoxy, amino, halo, nitro, cyano, and hydroxy. Aspect P20 is the compound of aspect P19, wherein R² is:

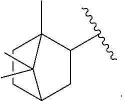 [SMILES: *C1CC2CCC1(C)C2(C)C]

Aspect P21 is directed to the compound of any one of aspects P1-P16, wherein R² is polyalkylene glycol. Aspect P22 is directed to the compound of aspect P21, wherein R² is polyethylene glycol. Aspect P23 is directed to the compound of any one of aspects P1-P22, wherein R³ is hydrogen. Aspect P24 is directed to the compound of any one of aspects P1-P22, wherein R³ is methyl. Aspect P25 is directed to the compound of any of claims P1-P16, wherein R² is C₁₋₁₂alkylene-methacrylate or is C₁₋₁₂alkylene-methacrylamide and R³ is hydrogen, wherein the C₁₋₁₂alkylene is substituted with zero, one or more groups independently selected from C₁-C₄ alkyl, C₁-C₆ alkoxy, phenoxy, amino, halo, nitro, cyano, and hydroxy. Aspect P26 is directed to the compound of aspect P25, wherein R² is:

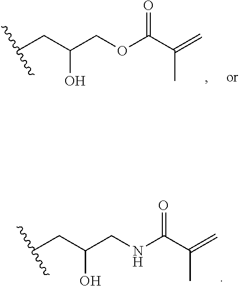 [SMILES: *CC(O)COC(=O)C(=C)C ; *CC(O)CNC(=O)C(=C)C]

Aspect P27 is directed to the compound of any one of aspects P1-P22, wherein X is —NH—. Aspect P28 is directed to the compound of any one of aspects P1-P22, wherein X is —O—. Aspect P29 is directed to the compound of aspect P1, selected from Table 1 hereinabove.

Aspect P30 is directed to a particle comprising: (a) a luminescent nanostructure; and (b) a ligand comprising at least one compound of any one of aspects P1-P29. Aspect P31 is directed to the particle of aspect P30, wherein the particle comprises a second ligand bound to the nanostructure, wherein the second ligand is a compound according to Formula (I) and is different from the first ligand. Aspect P32 is directed to the particle of aspect P31, wherein the particle comprises the following ligands:

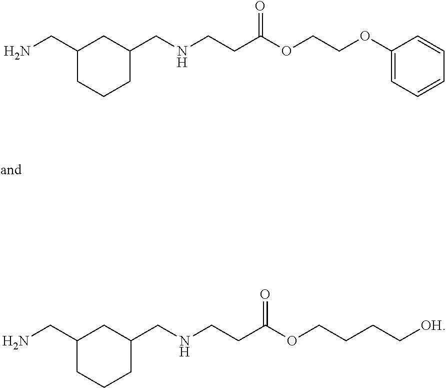 [SMILES: NCC1CCCC(CNCCC(=O)OCCOc2ccccc2)C1 ; NCC1CCCC(CNCCC(=O)OCCCCO)C1]

Aspect P33 is directed to the particle of any one of aspects P30-P32, wherein the luminescent nanostructure comprises Si, Ge, Sn, Se, Te, B, C, P, BN, BP, BAs, AlN, AlP, AlAs, AlSb, AgInS, AgGaS, AgInGaS, GaN, GaP, GaAs, GaSb, InN, InP, InAs, InSb, ZnO, ZnS, ZnSe, ZnTe, CdS, CdSe, CdSeZn, CdTe, HgS, HgSe, HgTe, BeS, BeSe, BeTe, MgS, MgSe, GeS, GeSe, GeTe, SnS, SnSe, SnTe, PbO, PbS, PbSe, PbTe, CuF, CuCi, CuBr, CuI, Si₃N₄, Ge₃N₄, Al₂O₃, Al₂CO, or a combination thereof. Aspect P34 is directed to the particle of aspect P33, wherein the luminescent nanostructure has a core-shell structure. Aspect P35 is directed to the particle of aspect P34, wherein the core comprises AgInGaS and the shell comprises GaS.

Aspect P36 is directed to a method of making a particle according to any one of aspects P30-P35, comprising mixing the luminescent nanostructure with the at least one compound of any one of aspects P1-P29.

Aspect P37 is directed to a method of making a population of particles, the method comprising: (a) mixing a population of luminescent nanostructures with a Michael-donor compound to form a mixture, wherein the Michael-donor compound is selected from a di-amine, a di-thiol or a compound having an amine group and a thiol group; (b) adding a Michael-acceptor compound to the mixture, wherein the Michael-acceptor compound comprises an acrylate, methacrylate, acrylamide or methacrylamide group; wherein a Michael reaction between the Michael-donor and Michael-acceptor compounds forms an adduct, and wherein the particles each comprise a luminescent nanostructure and a ligand, wherein the ligand comprises the adduct.

Aspect P38 is directed to a method of making a population of particles, the method comprising: (a) mixing a population of luminescent nanostructures with a Michael-acceptor compound to form a mixture, wherein the Michael-acceptor compound comprises an acrylate, methacrylate, acrylamide or methacrylamide group; (b) adding a Michael-donor compound to the mixture, wherein the Michael-donor compound is selected from a di-amine, a di-thiol or a compound having an amine group and a thiol group; wherein a Michael reaction between the Michael-donor and Michael-acceptor compounds forms an adduct, and wherein the particles each comprise a luminescent nanostructure and a ligand, wherein the ligand comprises the adduct.

Aspect P39 is directed to a method of making a population of particles according to aspect P36, the method comprising: (a) mixing a Michael-acceptor compound and a Michael-donor compound to form a mixture, wherein the Michael-donor compound is selected from a di-amine, a di-thiol or a compound having an amine group and a thiol group, and the Michael-acceptor compound comprises an acrylate, methacrylate, acrylamide or methacrylamide group, and wherein a Michael reaction between the Michael-donor and Michael-acceptor compounds forms an adduct; (b) adding a population of luminescent nanostructures to the mixture to form the population of particles; wherein the particles each comprise a luminescent nanostructure and a ligand, wherein the ligand comprises the adduct.

Aspect P40 is directed to the method of any one of aspects P37-P39, wherein the Michael-donor compound is a compound of Formula (II):

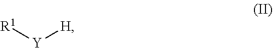

and the Michael-acceptor compound is a compound of Formula (III):

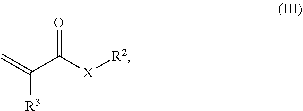

and wherein the adduct is according to Formula (I):

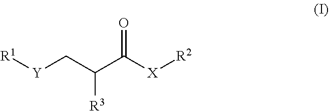

wherein the symbols R¹, R², R³, X, and Y are as defined hereinabove.

Aspect P41 is directed to a composition comprising: (a) a plurality of particles according to any one of aspects P30-P35, and/or made according to any one of aspects P36-P40; and (b) a carrier. Aspect P42 is directed to the composition of aspect P41, wherein the carrier additionally comprises triphenyl phosphite, pentaerythritol tetrakis[3-(3,5-di-tert-butyl-4-hydroxyphenyl)propionate, isopropylthioxanthone, 2,4-diethylthioxanthone, or 3-(trimethoxysilyl)propyl acrylate. Aspect P43 is directed to the composition of any one of aspects P41-P42, wherein the carrier is liquid. Aspect P44 is directed to the composition of aspect P43, wherein the carrier comprises a curable acrylate monomer. Aspect P45 is directed to the composition of any one of aspects P41-P42, wherein the carrier is solid. Aspect P46 is directed to the composition of aspect P45, wherein the carrier comprises a cured acrylate polymer. Aspect P47 is directed to the composition of any one of aspects P45-P46, wherein the composition is a film. Aspect P48 is directed to the composition of any one of aspects P45-P47, comprising: (a) a first region comprising a first population of the plurality of particles comprising first luminescent nanostructures for emitting light of a first colour, the first population of the plurality of particles dispersed in the carrier; and (b) a second region comprising a second population of the plurality of particles comprising second luminescent nanostructures for emitting light of a second colour different from the first colour, the second population of the plurality of particles dispersed in the carrier. Aspect P49 is directed to the composition of aspect P47 or P48, wherein the film exhibits a photon conversion efficiency (PCE) of from about 20% to about 35%. Aspect P50 is directed to the composition of aspect P49, wherein the film exhibits a PCE of from about 25% to about 30%. Aspect P51 is directed to a display device comprising the composition of any one of aspects P45-P50.

Aspect P52 is directed to a composition comprising: (a) a particle comprising a luminescent nanostructure and a ligand; and (b) a carrier comprising a curable acrylate monomer; and wherein the ligand comprises one or more acrylate, methacrylate, acrylamide or methacrylamide groups which are cross-linkable with the acrylate monomer of the carrier on curing.

Aspect P53 is directed to a composition comprising: (a) a particle comprising a luminescent nanostructure and a ligand; and (b) a carrier comprising an acrylate polymer; and wherein the ligand is cross-linked with the acrylate polymer of the carrier.

Aspect P54 is directed to a method of making a composition comprising: (a) mixing: (i) a particle comprising a luminescent nanostructure and a ligand comprising one or more of an acrylate, methacrylate, acrylamide or methacrylamide group, and (ii) a carrier comprising an acrylate monomer; and (b) curing at least some of the acrylate monomer of the carrier to form an acrylate polymer cross-linked with the ligand.

Aspect P55 is directed to a composition obtainable by the method of aspect P54.

Aspect P56 is directed to a method of fabricating the composition of aspect P47 or P48, comprising: depositing the composition of aspect P43 or P44 or P52; and then hardening and/or solidifying the composition.

Aspect P57 is directed to the method of aspect P56, wherein depositing comprises inkjet printing.

Aspect P58 is directed to a light source comprising: a first electrode; a second electrode; and a layer between the first electrode and the second electrode, the layer comprising particles according to any of claims 30 to 35 and/or obtainable according to any one of aspects P36-P40. Aspect P59 is directed to the light source of aspect P58, wherein the layer comprises the composition of any one of aspects P45-P50, or aspects P53-P55.

Aspect P60 is directed to an apparatus comprising: the composition of any one of aspects P45-P50, or P53 or P55; and a light source configured to emit light of a wavelength absorbed by the nanostructures. Aspect P61 is directed to the apparatus of aspect P60, comprising: a filter array comprising red light filters for transmitting red light, green light filters for transmitting green light, and blue light filters for transmitting blue light; and a light valve array. Aspect P62 is directed to the apparatus of aspect P60, comprising: a plurality of the light source and comprising the light source of aspect P60 or P61; and a plurality of the composition of any one of aspects P45-P50, or P53 or P55, wherein each of the plurality of the composition corresponds with, and is positioned to receive light emitted from, a respective light source of the plurality of the light source. Aspect P63 is directed to the apparatus of any one of aspects P60-P62, comprising: at least one processor; and at least one memory comprising computer program instructions, the at least one memory and the computer program instructions operable to, with the at least one processor, control the apparatus to output an image.

The above examples are to be understood as illustrative examples. It is to be understood that any feature described in relation to any one example may be used alone, or in combination with other features described, and may also be used in combination with one or more features of any other of the examples, or any combination of any other of the examples. Furthermore, equivalents and modifications not described above may also be employed without departing from the scope of the accompanying claims.

1. A compound comprising a structure: 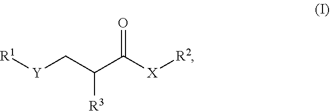
wherein:
Y is —NH— or —S—, and R¹ is selected from the group consisting of:
(i) Z—(C₁-C₆ alkylene)(C₃-C₈ cycloalkylene)(C₁-C₆ alkylene)-,
(ii) Z—(C₁-C₆ alkylene)(4- to 7-membered heterocyclylene)(C₁-C₆ alkylene)-,
(iii) Z—(C₁-C₆ alkylene)(C₆-C₁₄ arylene)(C₁-C₆ alkylene)-,
(iv) Z—(C₃-C₈ cycloalkylene)(C₁-C₆ alkylene)(C₃-C₈ cycloalkylene)-,
(v) polyalkyleneiminyl, and
(vi) Z—(C₁₋₁₂ alkylene)-; or
Y is absent, R¹ is (vii) Z—(C₁-C₆ alkylene)(4- to 7-membered heterocyclylene)-, and a nitrogen atom in the heterocyclylene provides a point of attachment of R¹ to a carbon in (I) which is β to a carbonyl;
wherein Z is an amino or —SH; and
wherein:
R³ is selected from the group consisting of hydrogen and C₁-C₆ alkyl, and R² is selected from the group consisting of:
(i) C₁-C₁₂ alkyl, substituted with zero, one, two, or three groups independently selected from C₁-C₄ alkyl, C₁-C₆ alkoxy, phenoxy, amino, halo, nitro, cyano, and hydroxy;
(ii) C₃-C₈ cycloalkyl, substituted with zero, one, two, or three groups independently selected from C₁-C₄ alkyl, C₁-C₆ alkoxy, amino, halo, nitro, cyano, and hydroxy; and
(iii) polyalkylene glycol, wherein zero, one or more units of the polyalkylene glycol chain is substituted with one or two C₁-C₄ alkyl groups; or
R² is (iv) C₁₋₁₂alkylene-methacrylate or C₁₋₁₂alkylene-methacrylamide, and R³ is hydrogen, the C₁₋₁₂alkylene is substituted with zero, one or more groups independently selected from C₁-C₄ alkyl, C₁-C₆ alkoxy, phenoxy, amino, halo, nitro, cyano, and hydroxy; and
wherein X is —NH— or —O—.

2. The compound of claim 1, wherein Z is amino and Y is —NH—.

3. A particle comprising:
a luminescent nanostructure; and
a ligand comprising the compound of claim 1.

4. The particle of claim 3, wherein the particle comprises a second ligand bound to the nanostructure, and wherein the second ligand is a compound according to Formula (I) which is different from the first ligand.

5. The particle of claim 4, wherein the particle comprises ligands

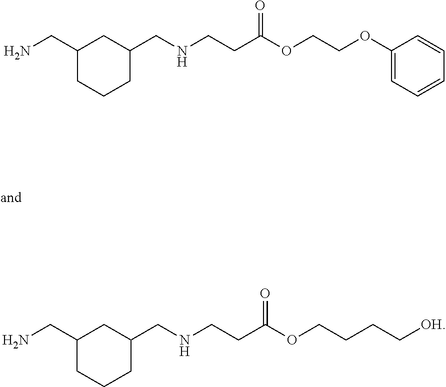 [SMILES: NCC1CCCC(CNCCC(=O)OCCOc2ccccc2)C1 ; NCC1CCCC(CNCCC(=O)OCCCCO)C1]

6. The particle of claim 3, wherein the luminescent nanostructure comprises Si, Ge, Sn, Se, Te, B, C, P, BN, BP, BAs, AlN, AlP, AlAs, AlSb, AgInS, AgGaS, AgInGaS, GaN, GaP, GaAs, GaSb, InN, InP, InAs, InSb, ZnO, ZnS, ZnSe, ZnTe, CdS, CdSe, CdSeZn, CdTe, HgS, HgSe, HgTe, BeS, BeSe, BeTe, MgS, MgSe, GeS, GeSe, GeTe, SnS, SnSe, SnTe, PbO, PbS, PbSe, PbTe, CuF, CuCl, CuBr, CuI, Si₃N₄, Ge₃N₄, Al₂O₃, Al₂CO, or a combination thereof.

7. The particle of claim 6, wherein the luminescent nanostructure has a core-shell structure.

8. The particle of claim 7, wherein the core comprises AgInGaS and the shell comprises GaS.

9. A method of making a particle comprising a luminescent nanostructure and a ligand comprising a compound comprising a structure 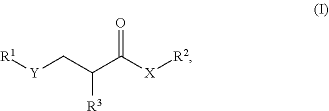
wherein
Y is —NH— or —S—, and R¹ is selected from the group consisting of:
(i) Z—(C₁-C₆ alkylene)(C₃-C₈ cycloalkylene)(C₁-C₆ alkylene)-,
(ii) Z—(C₁-C₆ alkylene)(4- to 7-membered heterocyclylene)(C₁-C₆ alkylene)-,
(iii) Z—(C₁-C₆ alkylene)(C₆-C₁₄ arylene)(C₁-C₆ alkylene)-,
(iv) Z—(C₃-C₈ cycloalkylene)(C₁-C₆ alkylene)(C₃-C₈ cycloalkylene)-,
(v) polyalkyleneiminyl, and
(vi) Z—(C₁₋₁₂ alkylene)-; or
Y is absent, R¹ is (vii) Z—(C₁-C₆ alkylene)(4- to 7-membered heterocyclylene)-, and a nitrogen atom in the heterocyclylene provides a point of attachment of R¹ to a carbon in (I) which is β to a carbonyl;
wherein Z is an amino or —SH; and
wherein
R³ is selected from the group consisting of hydrogen and C₁-C₆ alkyl, and R² is selected from the group consisting of:
(i) C₁-C₁₂ alkyl, substituted with zero, one, two, or three groups independently selected from C₁-C₄ alkyl, C₁-C₆ alkoxy, phenoxy, amino, halo, nitro, cyano, and hydroxy;
(ii) C₃-C₈ cycloalkyl, substituted with zero, one, two, or three groups independently selected from C₁-C₄ alkyl, C₁-C₆ alkoxy, amino, halo, nitro, cyano, and hydroxy; and
(iii) polyalkylene glycol, wherein zero, one or more units of the polyalkylene glycol chain is substituted with one or two C₁-C₄ alkyl groups; or
R² is (iv) C₁₋₁₂alkylene-methacrylate or C₁₋₁₂alkylene-methacrylamide, and R³ is hydrogen, the C₁₋₁₂alkylene is substituted with zero, one or more groups independently selected from C₁-C₄ alkyl, C₁-C₆ alkoxy, phenoxy, amino, halo, nitro, cyano, and hydroxy; and
wherein X is —NH— or —O—, the method comprising:
mixing the luminescent nanostructure with the compound.

10-11. (canceled)

12. A method of making a population of particles according to claim 9, the method comprising:
mixing a Michael-acceptor compound and a Michael-donor compound to form a mixture, wherein the Michael-donor compound is selected from a di-amine, a di-thiol or a compound having an amine group and a thiol group, and the Michael-acceptor compound comprises an acrylate, methacrylate, acrylamide or methacrylamide group, and wherein a Michael reaction between the Michael-donor and Michael-acceptor compounds forms an adduct; and
adding a population of luminescent nanostructures to the mixture to form the population of particles,
wherein the particles each comprise a luminescent nanostructure and a ligand, wherein the ligand comprises the adduct.

13. (canceled)

14. A composition comprising:
a plurality of particles according to claim 3; and
a carrier.

15. The composition of claim 14, wherein the composition is a film.

16. The composition of claim 14, comprising:
a first region comprising a first population of the plurality of particles comprising first luminescent nanostructures for emitting light of a first colour, the first population of the plurality of particles dispersed in the carrier; and
a second region comprising a second population of the plurality of particles comprising second luminescent nanostructures for emitting light of a second colour different from the first colour, the second population of the plurality of particles dispersed in the carrier.

17. The composition of claim 15, wherein the film exhibits a photon conversion efficiency (PCE) of from 20% to 35%.

18.-19. (canceled)
